(19) United States
(12) Patent Application Publication
(10) Pub. No.: US 2024/0115314 A1
(43) Pub. Date: Apr. 11, 2024

(54) ENERGY DELIVERY SYSTEMS WITH ABLATION INDEX

(71) Applicant: Acutus Medical, Inc., Carlsbad, CA (US)

(72) Inventors: Ahmad FALAHATPISHEH, San Marcos, CA (US); Steven YON, San Diego, CA (US); Xinwei SHI, San Diego, CA (US); Haizhe ZHANG, San Diego, CA (US); Derrick CHOU, San Diego, CA (US); Timothy CORVI, San Diego, CA (US); Graydon Earnest BEATTY, Bloomington, MN (US); Daniel WELSH, Encinitas, CA (US); Alexander HIGGINS, San Diego, CA (US); Marcus JULIAN, Vista, CA (US); R. Maxwell FLAHERTY, Topsfield, MA (US); J. Christopher FLAHERTY, Nottingham, NH (US)

(21) Appl. No.: 18/275,501

(22) PCT Filed: Feb. 17, 2022
(86) PCT No.: PCT/US2022/016722

§ 371 Date: Aug. 2, 2023

Related U.S. Application Data
(60) Provisional application No. 63/150,555, filed on Feb. 17, 2021.

(51) Int. Cl. A61B 18/14; A61B 90/00

(52) U.S. Cl. CPC A61B 18/1492; A61B 90/06; A61B 2090/065

(57) ABSTRACT

Provided herein are systems, devices, and methods for performing a medical procedure on a patient. The system comprises an ablation catheter comprising a shaft, at least one therapeutic element positioned on the shaft, and one or more tissue-contact sensing components configured to provide a contact signal related to the level of contact between the at least one therapeutic element and tissue. The system further comprises: an energy delivery unit configured to deliver ablative energy to the at least one therapeutic element to create a lesion in target tissue, and to provide an energy delivery data; a force module configured to receive the contact signal and provide contact data; and a processing unit configured to determine an ablation index based on both the energy delivery data and the contact data. The ablation index can represent the status of the lesion being created.

### RELATED APPLICATIONS

The present application claims priority to U.S. Provisional Patent Application Ser. No. 63/150,555, entitled “ENERGY DELIVERY SYSTEMS WITH ABLATION INDEX,” filed Feb. 17, 2021, the contents of which are hereby incorporated by reference.

The present application, while not claiming priority to, may be related to U.S. application Ser. No. 16/335,893, entitled “Ablation System with Force Control”, filed Mar. 22, 2019, which is a 35 USC 371 national stage filing of Patent Cooperation Treaty Application No. PCT/US2017/056064, entitled “Ablation System with Force Control”, filed Oct. 11, 2017, published as WO2018/071490, which claims priority to U.S. Provisional Application Ser. No. 62/406,748, entitled “Ablation System with Force Control”, filed Oct. 11, 2016, and U.S. Provisional Application Ser. No. 62/504,139, entitled “Ablation System with Force Control”, filed May 20, 2017, each of which is hereby incorporated by reference.

The present application, while not claiming priority to, may be related to U.S. application Ser. No. 16/097,955, entitled “Cardiac Information Dynamic Display System and Method”, filed Oct. 31, 2018, which is a 35 USC 371 national stage filing of Patent Cooperation Treaty Application No. PCT/US2017/030915, entitled “Cardiac Information Dynamic Display System and Method”, filed May 3, 2017, published as WO2017/192769, which claims priority to U.S. Provisional Application Ser. No. 62/331,351, entitled “Cardiac Information Dynamic Display System and Method”, filed May 3, 2016, each of which is hereby incorporated by reference.

The present application, while not claiming priority to, may be related to U.S. patent application Ser. No. 16/861,814, entitled “Catheter, System and Methods of Medical Uses of Same, including Diagnostic and Treatment Uses for the Heart”, filed Apr. 29, 2020, which is a continuation of U.S. Pat. No. 10,667,753, entitled “Catheter, System and Methods of Medical Uses of Same, Including Diagnostic and Treatment Uses for the Heart”, filed Jun. 19, 2018, which is a continuation of U.S. Pat. No. 10,004,459, entitled “Catheter, System and Methods of Medical Uses of Same, Including Diagnostic and Treatment Uses for the Heart”, filed Feb. 20, 2015, which is a 35 USC 371 national stage filing of Patent Cooperation Treaty Application No. PCT/US2013/057579, entitled “Catheter System and Methods of Medical Uses of Same, Including Diagnostic and Treatment Uses for the Heart”, filed Aug. 30, 2013, published as WO2014/036439, which claims priority to U.S. Patent Provisional Application Ser. No. 61/695,535, entitled “System and Method for Diagnosing and Treating Heart Tissue”, filed Aug. 31, 2012, each of which is hereby incorporated by reference.

The present application, while not claiming priority to, may be related to U.S. patent application Ser. No. 16/242,810, entitled “Expandable Catheter Assembly with Flexible Printed Circuit Board (PCB) Electrical Pathways”, filed Jan. 8, 2019, which is a continuation of U.S. Pat. No. 10,201,311, entitled “Expandable Catheter Assembly with Flexible Printed Circuit Board (PCB) Electrical Pathways”, filed Jul. 23, 2015, Which is a 35 USC 371 national stage filing of Patent Cooperation Treaty Application No. PCT/US2014/015261, entitled “Expandable Catheter Assembly with Flexible Printed Circuit Board (PCB) Electrical Pathways”, filed Feb. 7, 2014, published as WO2014/124231, which claims priority to U.S. Patent Provisional Application Ser. No. 61/762,363, entitled “Expandable Catheter Assembly with Flexible Printed Circuit Board (PCB) Electrical Pathways”, filed Feb. 8, 2013, each of which is hereby incorporated by reference.

The present application, while not claiming priority to, may be related to U.S. patent application Ser. No. 16/533,028, entitled “Method and Device for Determining and Presenting Surface Charge and Dipole Densities on Cardiac Walls”, filed Aug. 6, 2019, which is a continuation of U.S. Pat. No. 10,413,206, entitled “Method and Device for Determining and Presenting Surface Charge and Dipole Densities on Cardiac Walls”, filed Jun. 21, 2018, which is a continuation of U.S. Pat. No. 10,376,171, entitled “Method and Device for Determining and Presenting Surface Charge and Dipole Densities on Cardiac Walls”, filed Feb. 17, 2017, which is a continuation of U.S. Pat. No. 9,610,024, entitled “Method and Device for Determining and Presenting Surface Charge and Dipole Densities on Cardiac Walls”, filed Sep. 25, 2015, which is a continuation of U.S. Pat. No. 9,167,982, entitled “Method and Device for Determining and Presenting Surface Charge and Dipole Densities on Cardiac Walls”, filed Nov. 19, 2014, which is a continuation of U.S. Pat. No. 8,918,158 (hereinafter the '158 patent), entitled “Method and Device for Determining and Presenting Surface Charge and Dipole Densities on Cardiac Walls”, issued Dec. 23, 2014, which is a continuation of U.S. Pat. No. 8,700,119 (hereinafter the '119 patent), entitled “Method and Device for Determining and Presenting Surface Charge and Dipole Densities on Cardiac Walls”, issued Apr. 15, 2014, which is a continuation of U.S. Pat. No. 8,417,313 (hereinafter the '313 patent), entitled “Method and Device for Determining and Presenting Surface Charge and Dipole Densities on Cardiac Walls”, issued Apr. 9, 2013, which was a 35 USC 371 national stage filing of PCT Application No. PCT/CH2007/000380, entitled “Method and Device for Determining and Presenting Surface Charge and Dipole Densities on Cardiac Walls”, filed Aug. 3, 2007, published as WO2008/014629, which claimed priority to Swiss Patent Application No. 1251/06 filed Aug. 3, 2006, each of which is hereby incorporated by reference.

The present application, while not claiming priority to, may be related to U.S. patent application Ser. No. 16/568,768, entitled “Device and Method for the Geometric Determination of Electrical Dipole Densities on the Cardiac Wall”, filed Sep. 12, 2019, which is a continuation of U.S. Pat. No. 10,463,267, entitled “Device and Method for the Geometric Determination of Electrical Dipole Densities on the Cardiac Wall”, filed Jan. 29, 2018, which is a continuation of U.S. Pat. No. 9,913,589, entitled “Device and Method for the Geometric Determination of Electrical Dipole Densities on the Cardiac Wall”, filed Dec. 25, 2016, which is a continuation of U.S. Pat. No. 9,504,395, entitled “Device and Method for the Geometric Determination of Electrical Dipole Densities on the Cardiac Wall”, filed Oct. 19, 2015, which is a continuation of U.S. Pat. No. 9,192,318, entitled “Device and Method for the Geometric Determination of Electrical Dipole Densities on the Cardiac Wall”, filed Jul. 19, 2013, which is a continuation of U.S. Pat. No. 8,512,255, entitled “Device and Method for the Geometric Determination of Electrical Dipole Densities on the Cardiac Wall”, issued Aug. 20, 2013, published as US2010/0298690 (hereinafter the '690 publication), which was a 35 USC 371 national stage application of Patent Cooperation Treaty Application No. PCT/IB2009/000071 filed Jan. 16, 2009, entitled “A Device and Method for the Geometric Determination of Electrical Dipole Densities on the Cardiac Wall”, published as WO2009/090547, which claimed priority to Swiss Patent Application 00068/08 filed Jan. 17, 2008, each of which is hereby incorporated by reference.

The present application, while not claiming priority to, may be related to U.S. patent application Ser. No. 17/673,995, entitled “Device and Method for the Geometric Determination of Electrical Dipole Densities on the Cardiac Wall”, filed Feb. 17, 2022, which is a continuation of U.S. patent application Ser. No. 16/389,006, entitled “Device and Method for the Geometric Determination of Electrical Dipole Densities on the Cardiac Wall”, filed Apr. 19, 2019, which is a continuation of U.S. Pat. No. 10,314,497, entitled “Device and Method for the Geometric Determination of Electrical Dipole Densities on the Cardiac Wall”, filed Mar. 20, 2018, which is a continuation of U.S. Pat. No. 9,968,268, entitled “Device and Method for the Geometric Determination of Electrical Dipole Densities on the Cardiac Wall”, filed Aug. 8, 2017, which is a continuation of U.S. Pat. No. 9,757,044, entitled “Device and Method for the Geometric Determination of Electrical Dipole Densities on the Cardiac Wall”, filed Sep. 6, 2013, which is a 35 USC 371 national stage filing of Patent Cooperation Treaty Application No. PCT/US2012/028593, entitled “Device and Method for the Geometric Determination of Electrical Dipole Densities on the Cardiac Wall”, published as WO2012/122517 (hereinafter the '517 publication), which claimed priority to U.S. Patent Provisional Application Ser. No. 61/451,357, each of which is hereby incorporated by reference.

The present application, while not claiming priority to, may be related to U.S. Design Pat. No. 29/681,827, entitled “Set of Transducer-Electrode Pairs for a Catheter”, filed Feb. 28, 2019, which is a divisional of U.S. Design Pat. No. D851,774, entitled “Set of Transducer-Electrode Pairs for a Catheter”, filed Feb. 6, 2017, which is a divisional of U.S. Design Pat. No. D782,686, entitled “Transducer-Electrode Pair for a Catheter”, filed Dec. 2, 2013, which is a 35 USC 371 national stage filing of Patent Cooperation Treaty Application No. PCT/US2013/057579, entitled “Catheter System and Methods of Medical Uses of Same, Including Diagnostic and Treatment Uses for the Heart”, filed Aug. 30, 2013, which claims priority to U.S. Patent Provisional Application Ser. No. 61/695,535, entitled “System and Method for Diagnosing and Treating Heart Tissue”, filed Aug. 31, 2012, each of which is hereby incorporated by reference.

The present application, while not claiming priority to, may be related to U.S. patent application Ser. No. 16/111,538, entitled “Gas-Elimination Patient Access Device”, filed Aug. 24, 2018, which is a continuation of U.S. Pat. No. 10,071,227, entitled “Gas-Elimination Patient Access Device”, filed Jul. 14, 2016, which is a 35 USC 371 national stage filing of Patent Cooperation Treaty Application No. PCT/US2015/11312, entitled “Gas-Elimination Patient Access Device”, filed Jan. 14, 2015, which claims priority to U.S. Patent Provisional Application Ser. No. 61/928,704, entitled “Gas-Elimination Patient Access Device”, filed Jan. 17, 2014, which is hereby incorporated by reference.

The present application, while not claiming priority to, may be related to U.S. patent application Ser. No. 17/578,522, entitled “Cardiac Analysis User Interface System and Method”, filed Jan. 19, 2022, which is a continuation of U.S. application Ser. No. 15/128,563, entitled “Cardiac Analysis User Interface System and Method”, filed Sep. 23, 2016, which is a 35 USC 371 national stage filing of Patent Cooperation Treaty Application No. PCT/US2015/22187, entitled “Cardiac Analysis User Interface System and Method”, filed Mar. 24, 2015, which claims priority to U.S. Patent Provisional Application Ser. No. 61/970,027, entitled “Cardiac Analysis User Interface System and Method”, filed Mar. 28, 2014, which is hereby incorporated by reference.

The present application, while not claiming priority to, may be related to U.S. patent application Ser. No. 17/063,901, entitled “Devices and Methods for Determination of Electrical Dipole Densities on a Cardiac Surface”, filed Oct. 6, 2020, which is a continuation of U.S. Pat. No. 10,828,011, entitled “Devices and Methods for Determination of Electrical Dipole Densities on a Cardiac Surface”, filed Mar. 2, 2016, which is a 35 USC 371 national stage filing of Patent Cooperation Treaty Application No. PCT/US2014/54942, entitled “Devices and Methods for Determination of Electrical Dipole Densities on a Cardiac Surface”, filed Sep. 10, 2014, which claims priority to U.S. Patent Provisional Application Ser. No. 61/877,617, entitled “Devices and Methods for Determination of Electrical Dipole Densities on a Cardiac Surface”, filed Sep. 13, 2013, which is hereby incorporated by reference.

The present application, while not claiming priority to, may be related to U.S. patent application Ser. No. 16/849,045, entitled “Localization System and Method Useful in the Acquisition and Analysis of Cardiac Information”, filed Apr. 15, 2020, which is a continuation of U.S. Pat. No. 10,653,318, entitled “Localization System and Method Useful in the Acquisition and Analysis of Cardiac Information”, filed Oct. 26, 2017, which is a 35 USC 371 national stage filing of Patent Cooperation Treaty Application No. PCT/US2016/032420, entitled “Localization System and Method Useful in the Acquisition and Analysis of Cardiac Information”, filed May 13, 2016, which claims priority to U.S. Patent Provisional Application Ser. No. 62/161,213, entitled “Localization System and Method Useful in the Acquisition and Analysis of Cardiac Information”, filed May 13, 2015, which is hereby incorporated by reference.

The present application, while not claiming priority to, may be related to U.S. patent application Ser. No. 15/569,231, entitled “Cardiac Virtualization Test Tank and Testing System and Method”, filed Oct. 25, 2017, which is a 35 USC 371 national stage filing of Patent Cooperation Treaty Application No. PCT/US2016/031823, filed May 11, 2016, which claims priority to U.S. Patent Provisional Application Ser. No. 62/160,501, entitled “Cardiac Virtualization Test Tank and Testing System and Method”, filed May 12, 2015, which is hereby incorporated by reference.

The present application, while not claiming priority to, may be related to U.S. patent application Ser. No. 15/569,185, entitled “Cardiac Virtualization Test Tank and Testing System and Method”, filed Oct. 25, 2017, which is a 35 USC 371 national stage filing of Patent Cooperation Treaty Application No. PCT/US2016/032017, filed May 12, 2016, which claims priority to U.S. Patent Provisional Application Ser. No. 62/160,529, entitled “Ultrasound Sequencing System and Method”, filed May 12, 2015, which is hereby incorporated by reference.

The present application, while not claiming priority to, may be related to U.S. patent application Ser. No. 16/097,959, entitled “Cardiac Mapping System with Efficiency Algorithm”, filed Oct. 31, 2018, which is a 35 USC 371 national stage filing of Patent Cooperation Treaty Application No. PCT/US2017/030922, entitled “Cardiac Mapping System with Efficiency Algorithm”, filed May 3, 2017, which claims priority to U.S. Patent Provisional Application Ser. No. 62/413,104, entitled “Cardiac Mapping System with Efficiency Algorithm”, filed Oct. 26, 2016, which is hereby incorporated by reference.

The present application, while not claiming priority to, may be related to U.S. patent application Ser. No. 16/961,809, entitled “System for Identifying Cardiac Conduction Patterns”, filed Jul. 13, 2020, which is a 35 USC 371 national stage filing of Patent Cooperation Treaty Application No. PCT/US2019/014498, entitled “System for Identifying Cardiac Conduction Patterns”, filed Jan. 22, 2019, which claims priority to U.S. Patent Provisional Application Ser. No. 62/619,897, entitled “System for Recognizing Cardiac Conduction Patterns”, filed Jan. 21, 2018, and U.S. Patent Provisional Application Ser. No. 62/668,647, entitled “System for identifying Cardiac Conduction Patterns”, filed May 8, 2018, each of which is hereby incorporated by reference.

The present application, while not claiming priority to, may be related to U.S. patent application Ser. No. 17/048,151, entitled “Cardiac Information Processing System”, filed Oct. 16, 2020, which is a 35 USC 371 national stage filing of Patent Cooperation Treaty Application No. PCT/US2019/031131, entitled “Cardiac Information Processing System”, filed May 7, 2019, which claims priority to U.S. Provisional Application Ser. No. 62/668,659, entitled “Cardiac Information Processing System”, filed May 8, 2018, and U.S. Patent Provisional Application Ser. No. 62/811,735, entitled “Cardiac information Processing System”, filed Feb. 28, 2019, each of which is hereby incorporated by reference.

The present application, while not claiming priority to, may be related to Patent Cooperation Treaty Application No. PCT/US2019/060433, entitled “Systems and Methods for Calculating Patient Information”, filed Nov. 8, 2019, which claims priority to U.S. Provisional Application Ser. No. 62/757,961, entitled “Systems and Methods for Calculating Patient Information”, filed Nov. 9, 2018, each of which is hereby incorporated by reference.

The present application, while not claiming priority to, may be related to U.S. patent application Ser. No. 17/601,661, entitled “Cardiac Information Processing System”, filed Oct. 5, 2021, which is a 35 USC 371 national stage filing of Patent Cooperation Treaty Application No. PCT/US2020/028779, entitled “System for Creating a Composite Map”, filed Apr. 17, 2020, which claims priority to U.S. Provisional Application Ser. No. 62/835,538, entitled “System for Creating a Composite Map”, filed Apr. 18, 2019, and U.S. Provisional Application Ser. No. 62/925,030, entitled “System for Creating a Composite Map”, filed Oct. 23, 2019, each of which is hereby incorporated by reference.

The present application, while not claiming priority to, may be related to U.S. patent application Ser. No. 17/613,249, entitled “Cardiac Information Processing System”, filed Nov. 22, 2021, which is a 35 USC 371 national stage filing of Patent Cooperation Treaty Application No. PCT/US2020/036110, entitled “Systems and Methods for Performing Localization Within a Body”, filed Jun. 4, 2020, which claims priority to U.S. Provisional Application Ser. No. 62/857,055, entitled “Systems and Methods for Performing Localization Within a Body”, filed Jun. 4, 2019, each of which is hereby incorporated by reference.

The present application, while not claiming priority to, may be related to Patent Cooperation Treaty Application No. PCT/US2020/061458, entitled “Tissue Treatment Systems, Devices, and Methods”, filed Nov. 20, 2020, which claims priority to U.S. Provisional Application Ser. No. 62/939,412, entitled “Tissue Treatment Systems, Devices, and Methods”, filed Nov. 22, 2019, and U.S. Provisional Application Ser. No. 63/175,280, entitled “Tissue Treatment Systems, Devices, and Methods”, filed Sep. 7, 2020, each of which is hereby incorporated by reference.

### FIELD OF THE INVENTION

The present inventive concepts relate generally to systems, devices, and methods for ablating tissue, and in particular, for ablating tissue of a patient's heart.

### BACKGROUND

Various systems for ablating tissue of a patient are commercially available. There are various drawbacks to the current systems, including the ability of a clinician operator to properly assess the status of tissue receiving ablative energy. There is a need for improved systems, methods, and devices for ablating tissue of a patient.

### SUMMARY

According to a first aspect of the present inventive concepts, a system for performing a medical procedure on a patient, comprising: an ablation catheter comprises: a shaft; at least one therapeutic element positioned on the shaft; and one or more tissue-contact sensing components configured to provide a contact signal related to the level of contact between the at least one therapeutic element and tissue; an energy delivery unit configured to deliver ablative energy to the at least one therapeutic element to create a lesion in target tissue, and to provide an energy delivery data related to one or more parameters of the delivered energy; a force module configured to receive the contact signal and provide contact data related to the level of contact between the at least one therapeutic element and tissue; and a processing unit configured to receive the energy delivery data and the contact data and to determine an ablation index based on both the energy delivery data and the contact data. The ablation index can represent the status of the lesion being created. The ablation index can be based on the following calculations:

W(t,\hat{P},\hat{F})=κ+W_{∞} ln(αt+1)ln(β\hat{P}+1)(1−exp(−γ\hat{F}))

with

$$\begin{matrix}\hat{P} = {{\int }^{T}}_{{t}_{0}} P ( t ) dt \\ \hat{F} = \frac{1}{( T - {t}_{0} )} {\int }_{{t}_{0}}^{T} F ( t ) dt or \hat{F} = {\int }_{{t}_{0}}^{T} F ( t ) dt\end{matrix}$$

where t₀ is time at the start of ablation. The parameters κ, W_{∞}, α, β, γ can be obtained via an algorithm using an iterated non-linear least squares technique.

According to another aspect of the present inventive concepts, a system for performing a medical procedure on a patient, comprises: an ablation catheter comprising: a shaft; at least one therapeutic element positioned on the shaft; and one or more tissue-contact sensing components configured to provide a contact signal related to the level of contact between the at least one therapeutic element and tissue; an energy delivery unit configured to deliver ablative energy to the at least one therapeutic element to create a lesion in target tissue, and to provide an energy delivery data related to one or more parameters of the delivered energy; a force module configured to receive the contact signal and provide contact data related to the level of contact between the at least one therapeutic element and tissue; and a processing unit configured to receive the energy delivery data and the contact data and to determine an ablation index based on both the energy delivery data and the contact data. The ablation index can represent the status of the lesion being created. The ablation index can be based on a predictive model, such that the model is described by:

$${W}_{p} = α ln ( β \hat{P} + 1 ) + γ \\ \hat{P} = \int P \hat{F} dt \\ \hat{F} = \{ \begin{matrix}\frac{F}{10} : & F \leq 10 \\ 1 : & F > 10\end{matrix}$$

where F is in grams, and power, time, and lesion size are in units consistent with input data, and such that the model includes three empirical constants that are determined by a non-linear recursive least squares method, α, β, and γ.

In some embodiments, the at least one therapeutic element comprises an electrode positioned on a distal tip of the shaft.

In some embodiments, the contact data includes a bias related to the angle of contact between the at least one therapeutic element and the tissue. The contact data can be scaled based on the contact angle. The contact data can be scaled using the following equation:

\hat{F}ₛ=\hat{F}(C₁ cos²(θ)+C₂ sin²(θ))

where θ is the contact angle relative to the tissue surface and the two constants C₁ and C₂ are determined empirically by evaluation of the variation of contact force in a controlled environment.
In some embodiments, the system is configured to provide haptic information related to the ablation index to a user. The ablation catheter can further comprise a vibrational transducer configured to provide the haptic information to the user. The amplitude and/or frequency of the vibration of the vibrational transducer can change as the ablation index changes.

In some embodiments, the force module is configured to determine a contact state between the ablation catheter and a cardiac wall by implementing k-Mean clustering of measurements made in 4D space.

In some embodiments, the ablation catheter comprises three or more electrodes, and the force module is configured to determine a contact state between the ablation catheter and a cardiac wall based on a resistor network model. The force module can be further configured to build a series of non-linear equations based on the model and to solve the equations for the resistance values between two of the three or more electrodes of the ablation catheter.

In some embodiments, the ablation catheter comprises tip electrode (1), source electrode (2), and sink electrode (3), and a quantity κ is used to detect a contact state of the ablation catheter, such that

$$κ = \frac{{\partial }^{2} V}{\partial {x}^{2}} {\vert}_{Elec \#2} = 2 [ \frac{{V}_{3} - {V}_{2} ( 1 + α ) + {αV}_{1}}{α ( 1 + α ) {h}^{2}} ]$$

where x is the coordinate along the axis of the ablation catheter; V₁, V₂, V₃ are voltage values at the tip, source, and sink electrodes in a 2-3 configuration; h=x₂−x₁ is the distance between the tip electrode to the second electrode (center-to-center); and α=(x₃−x₂)/(x₂−x₁) is the ratio of the distances among electrodes 1, 2, and 3.

In some embodiments, the system is configured to provide a therapeutic strategy to a user comprising a set of one or more energy delivery patterns collectively configured to address one or more conduction patterns identified by the system. A first set of energy delivery patterns can comprise an energy delivery pattern that creates a set of lesions in a rosette pattern. A first set of energy delivery patterns can create a first set of lesions, and a second set of energy delivery patterns can comprise a lesion segment that connects the first set of lesions to an anatomical structure and/or a second set of lesions. The second set of lesions can comprise an isolation line for one or more pulmonary veins.

The technology described herein, along with the attributes and attendant advantages thereof, will best be appreciated and understood in view of the following detailed description taken in conjunction with the accompanying drawings in which representative embodiments are described by way of example.

### INCORPORATION BY REFERENCE

All publications, patents, and patent applications mentioned in this specification are herein incorporated by reference to the same extent as if each individual publication, patent, or patent application was specifically and individually indicated to be incorporated by reference. The content of all publications, patents, and patent applications mentioned in this specification are herein incorporated by reference in their entirety for all purposes.

### BRIEF DESCRIPTION OF THE DRAWINGS

FIG. 1 illustrates a schematic view of a system for performing a medical procedure on a patient, consistent with the present inventive concepts.

FIGS. 2A and 2B illustrate a perspective view of the distal portion of an ablation catheter and a schematic view of a resistor network model, respectively, consistent with the present inventive concepts.

FIGS. 3A and 3B illustrate an indicator in a series of states and a set of indicators showing instantaneous information and information over time, respectively, consistent with the present inventive concepts.

FIG. 4 illustrates a method of providing a therapy, consistent with the present inventive concepts.

FIG. 5 illustrates a graph of voltages measured from electrodes of an ablation catheter, consistent with the present inventive concepts.

FIGS. 5A and 5B illustrate various graphs of test data collected by the applicant, consistent with the present inventive concepts.

FIGS. 6A-C illustrate a distal portion of an ablation catheter, a position indicating line, and a graph of voltage, respectively, consistent with the present inventive concepts.

FIGS. 6D-F illustrate various graphs of recorded electrode voltages and calculated data, consistent with the present inventive concepts.

FIGS. 7A-E illustrate various simulation schematics and results, consistent with the present inventive concepts.

FIG. 8 illustrates a graph of in-vitro study data, consistent with the present inventive concepts.

### DETAILED DESCRIPTION OF THE DRAWINGS

Reference will now be made in detail to the present embodiments of the technology, examples of which are illustrated in the accompanying drawings. Similar reference numbers may be used to refer to similar components. However, the description is not intended to limit the present disclosure to particular embodiments, and it should be construed as including various modifications, equivalents, and/or alternatives of the embodiments described herein.

It will be understood that the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) when used herein, specify the presence of stated features, integers, steps, operations, elements, and/or components, but do not preclude the presence or addition of one or more other features, integers, steps, operations, elements, components, and/or groups thereof.

It will be further understood that, although the terms first, second, third, etc. may he used herein to describe various limitations, elements, components, regions, layers and/or sections, these limitations, elements, components, regions, layers and/or sections should not be limited by these terms. These terms are only used to distinguish one limitation, element, component, region, layer or section from another limitation, element, component, region, layer or section. Thus, a first limitation, element, component, region, layer or section discussed below could be termed a second limitation, element, component, region, layer or section without departing from the teachings of the present application.

It will be further understood that when an element is referred to as being “on”, “attached”, “connected” or “coupled” to another element, it can be directly on or above, or connected or coupled to, the other element, or one or more intervening elements can be present. In contrast, when an element is referred to as being “directly on”, “directly attached”, “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

It will be further understood that when a first element is referred to as being “in” “on” and/or “within” a second element, the first element can be positioned: within an internal space of the second element, within a portion of the second element (e.g., within a wall of the second element); positioned on an external and/or internal surface of the second element; and combinations of one or more of these.

As used herein, the term “proximate”, when used to describe proximity of a first component or location to a second component or location, is to be taken to include one or more locations near to the second component or location, as well as locations in, on and/or within the second component or location. For example, a component positioned proximate an anatomical site (e.g., a target tissue location), shall include components positioned near to the anatomical site, as well as components positioned in, on and/or within the anatomical site.

Spatially relative terms, such as “beneath,” “below,” “lower,” “above,” “upper” and the like may be used to describe an element and/or feature's relationship to another element(s) and/or feature(s) as, for example, illustrated in the figures. It will be further understood that the spatially relative terms are intended to encompass different orientations of the device in use and/or operation in addition to the orientation depicted in the figures. For example, if the device in a figure is turned over, elements described as “below” and/or “beneath” other elements or features would then be oriented “above” the other elements or features. The device can be otherwise oriented (e.g., rotated 90 degrees or at other orientations) and the spatially relative descriptors used herein interpreted accordingly.

The terms “reduce”, “reducing”, “reduction” and the like, where used herein, are to include a reduction in a quantity, including a reduction to zero. Reducing the likelihood of an occurrence shall include prevention of the occurrence. Correspondingly, the terms “prevent”, “preventing”, and “prevention” shall include the acts of “reduce”, “reducing”, and “reduction”, respectively.

The term “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

The term “one or more”, where used herein can mean one, two, three, four, five, six, seven, eight, nine, ten, or more, up to any number.

The terms “and combinations thereof” and “and combinations of these” can each be used herein after a list of items that are to be included singly or collectively. For example, a component, process, and/or other item selected from the group consisting of: A; B; C; and combinations thereof, shall include a set of one or more components that comprise: one, two, three or more of item A; one, two, three or more of item B; and/or one, two, three, or more of item C.

In this specification, unless explicitly stated otherwise, “and” can mean “or”, and “or” can mean “and”. For example, if a feature is described as having A, B, or C, the feature can have A, B, and C, or any combination of A, B, and C. Similarly, if a feature is described as having A, B, and C, the feature can have only one or two of A, B, or C.

As used herein, when a quantifiable parameter is described as having a value “between” a first value X and a second value Y, it shall include the parameter having a value of: at least X, no more than Y, and/or at least X and no more than Y. For example, a length of between 1 and 10 shall include a length of at least 1 (including values greater than 10), a length of less than 10 (including values less than 1), and/or values greater than 1 and less than 10.

The expression “configured (or set) to” used in the present disclosure may be used interchangeably with, for example, the expressions “suitable for”, “having the capacity to”, “designed to”, “adapted to”, “made to” and “capable of” according to a situation. The expression “configured (or set) to” does not mean only “specifically designed to” in hardware. Alternatively, in some situations, the expression “a device configured to” may mean that the device “can” operate together with another device or component.

As used herein, the term “threshold” refers to a maximum level, a minimum level, and/or range of values correlating to a desired or undesired state. In some embodiments, a system parameter is maintained above a minimum threshold, below a maximum threshold, within a threshold range of values, and/or outside a threshold range of values, such as to cause a desired effect (e.g., efficacious therapy) and/or to prevent or otherwise reduce (hereinafter “prevent”) an undesired event (e.g., a device and/or clinical adverse event). In some embodiments, a system parameter is maintained above a first threshold (e.g., above a first temperature threshold to cause a desired therapeutic effect to tissue) and below a second threshold (e.g., below a second temperature threshold to prevent undesired tissue damage). In some embodiments, a threshold value is determined to include a safety margin, such as to account for patient variability, system variability, tolerances, and the like. As used herein, “exceeding a threshold” relates to a parameter going above a maximum threshold, below a minimum threshold, within a range of threshold values and/or outside of a range of threshold values.

As described herein, “room pressure” shall mean pressure of the environment surrounding the systems and devices of the present inventive concepts. Positive pressure includes pressure above room pressure or simply a pressure that is greater than another pressure, such as a positive differential pressure across a fluid pathway component such as a valve. Negative pressure includes pressure below room pressure or a pressure that is less than another pressure, such as a negative differential pressure across a fluid component pathway such as a valve. Negative pressure can include a vacuum but does not imply a pressure below a vacuum. As used herein, the term “vacuum” can be used to refer to a full or partial vacuum, or any negative pressure as described hereabove.

The term “diameter” where used herein to describe a non-circular geometry is to be taken as the diameter of a hypothetical circle approximating the geometry being described. For example, when describing a cross section, such as the cross section of a component, the term “diameter” shall be taken to represent the diameter of a hypothetical circle with the same cross sectional area as the cross section of the component being described.

The terms “major axis” and “minor axis” of a component where used herein are the length and diameter, respectively, of the smallest volume hypothetical cylinder which can completely surround the component.

As used herein, the term “functional element” is to be taken to include one or more elements constructed and arranged to perform a function. A functional element can comprise a sensor and/or a transducer. In some embodiments, a functional element is configured to deliver energy and/or otherwise treat tissue (e.g., a functional element configured as a treatment element). Alternatively or additionally, a functional element (e.g., a functional element comprising a sensor) can be configured to record one or more parameters, such as a patient physiologic parameter; a patient anatomical parameter (e.g., a tissue geometry parameter); a patient environment parameter; and/or a system parameter. In some embodiments, a sensor or other functional element is configured to perform a diagnostic function (e.g., to gather data used to perform a diagnosis). In some embodiments, a functional element is configured to perform a therapeutic function (e.g., to deliver therapeutic energy and/or a therapeutic agent). In some embodiments, a functional element comprises one or more elements constructed and arranged to perform a function selected from the group consisting of: deliver energy; extract energy (e.g., to cool a component); deliver a drug or other agent; manipulate a system component or patient tissue; record or otherwise sense a parameter such as a patient physiologic parameter or a system parameter; and combinations of one or more of these. A functional element can comprise a fluid and/or a fluid delivery system. A functional element can comprise a reservoir, such as an expandable balloon or other fluid-maintaining reservoir. A “functional assembly” can comprise an assembly constructed and arranged to perform a function, such as a diagnostic and/or therapeutic function. A functional assembly can comprise an expandable assembly. A functional assembly can comprise one or more functional elements.

The term “transducer” where used herein is to be taken to include any component or combination of components that receives energy or any input, and produces an output. For example, a transducer can include an electrode that receives electrical energy, and distributes the electrical energy to tissue (e.g., based on the size of the electrode). In some configurations, a transducer converts an electrical signal into any output, such as: light (e.g., a transducer comprising a light emitting diode or light bulb), sound (e.g., a transducer comprising a piezo crystal configured to deliver ultrasound energy); pressure (e.g., an applied pressure or force); heat energy; cryogenic energy; chemical energy; mechanical energy (e.g., a transducer comprising a motor or a solenoid); magnetic energy; and/or a different electrical signal (e.g., different than the input signal to the transducer). Alternatively or additionally, a transducer can convert a physical quantity (e.g., variations in a physical quantity) into an electrical signal. A transducer can include any component that delivers energy and/or an agent to tissue, such as a transducer configured to deliver one or more of: electrical energy to tissue (e.g., a transducer comprising one or more electrodes); light energy to tissue (e.g., a transducer comprising a laser, light emitting diode and/or optical component such as a lens or prism); mechanical energy to tissue (e.g., a transducer comprising a tissue manipulating element); sound energy to tissue (e.g., a transducer comprising a piezo crystal); chemical energy; electromagnetic energy; magnetic energy; and combinations of one or more of these.

As used herein, the term “fluid” can refer to a liquid, gas, gel, or any flowable material, such as a material which can be propelled through a lumen and/or opening.

As used herein, the term “material” can refer to a single material, or a combination of two, three, four, or more materials.

It is appreciated that certain features of the inventive concepts, which are, for clarity, described in the context of separate embodiments, may also be provided in combination in a single embodiment. Conversely, various features of the inventive concepts which are, for brevity, described in the context of a single embodiment, may also be provided separately or in any suitable sub-combination. For example, it will be appreciated that all features set out in any of the claims (whether independent or dependent) can be combined in any given way.

It is to be understood that at least some of the figures and descriptions of the inventive concepts have been simplified to focus on elements that are relevant for a clear understanding of the inventive concepts, while eliminating, for purposes of clarity, other elements that those of ordinary skill in the art will appreciate may also comprise a portion of the inventive concepts. However, because such elements are well known in the art, and because they do not necessarily facilitate a better understanding of the inventive concepts, a description of such elements is not provided herein.

Terms defined in the present disclosure are only used for describing specific embodiments of the present disclosure and are not intended to limit the scope of the present disclosure. Terms provided in singular forms are intended to include plural forms as well, unless the context clearly indicates otherwise. All of the terms used herein, including technical or scientific terms, have the same meanings as those generally understood by an ordinary person skilled in the related art, unless otherwise defined herein. Terms defined in a generally used dictionary should be interpreted as having meanings that are the same as or similar to the contextual meanings of the relevant technology and should not be interpreted as having ideal or exaggerated meanings, unless expressly so defined herein. In some cases, terms defined in the present disclosure should not be interpreted to exclude the embodiments of the present disclosure.

Provided herein are systems, devices, and methods for performing a medical procedure on a patient. A processing unit can be configured to receive energy delivery data and/or contact data from an ablation catheter and to determine an ablation index based on the data. In some embodiments, the ablation index comprises a value indicative of the efficacy of an ablation. The ablation index can represent the status of a lesion being created, for example an index displayed to a clinician while an ablation is being performed. The ablation index can be based on a predictive model, such as a model configured to incorporate force, power, time, and one or more empirical constants. The system can be configured to deliver one or more forms of energy to the ablation catheter to ablate tissue, such as radiofrequency (RF) energy and/or another energy form.

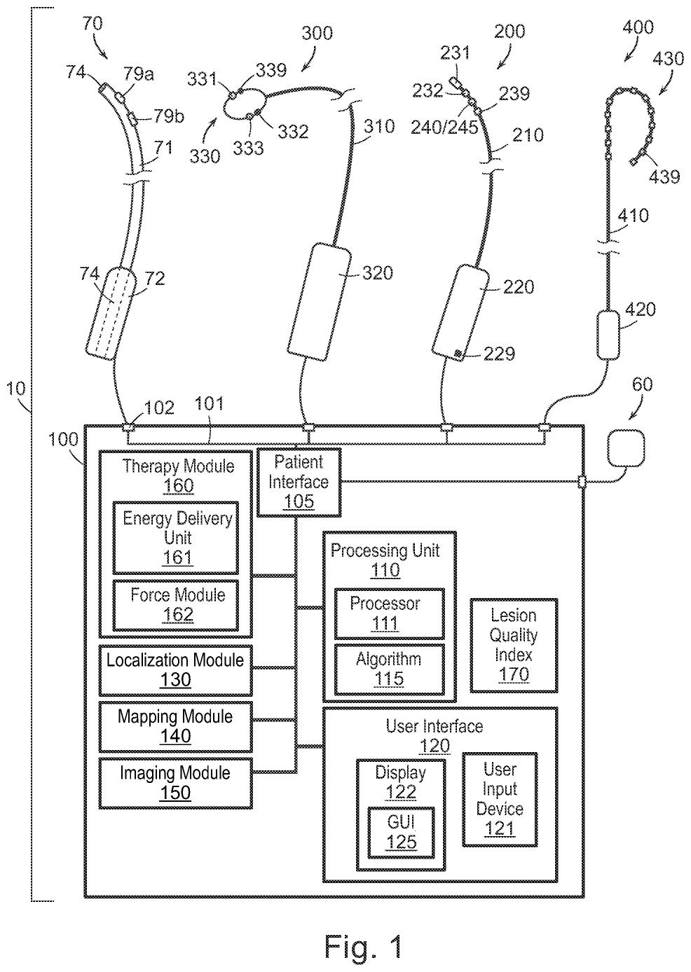

Referring now to FIG. 1, a schematic view of a system for performing a medical procedure on a patient (e.g., a human or other mammal) is illustrated, consistent with the present inventive concepts. The medical procedure can comprise a therapeutic procedure and/or a combined diagnostic and therapeutic procedure. System 10 can comprise console 100 that attaches to one, two, or more patient insertable devices, as well as receives and provides user inputs and outputs. System 10 can include one, two, or more treatment devices, ablation catheter 200 shown, which can comprise one or more ablation catheters. System 10 can also include one, two, or more mapping devices, mapping catheter 300 shown, one, two, or more patient introduction devices, sheath 70 shown, one, two, or more optional functional catheters, auxiliary catheter 400 shown, and/or one, two, or more patient patches, patch 60 shown. Console 100 operably attaches (e.g., electrically, mechanically, fluidly, sonically, and/or optically) to one or more devices 200, 300, 400, 70, and/or patches 60. Console 100 can be configured to deliver one, two, or more forms of energy (e.g., deliver energy to tissue via a catheter and/or other energy delivery device of system 10). As used herein, delivery of energy shall include the transfer of energy to tissue (e.g., to heat, ablate, cause the necrosis of, and/or otherwise affect tissue), as well as the extraction of energy from tissue (e.g., to cool, freeze, and/or cryogenically ablate tissue). System 10 can be configured to deliver energy in tissue to create a lesion in the tissue, such as to create one or more lesions in heart tissue to treat atrial fibrillation (AF) and/or another arrhythmia of the patient.

Ablation catheter 200 can comprise shaft 210, with handle 220 positioned on the proximal end of shaft 210. Ablation catheter 200 can comprise an electrode and/or other therapeutic element, tip electrode 231, positioned on a distal portion of shaft 210 (e.g., on the distal end of shaft 210 as shown). Tip electrode 231 can be configured to deliver radiofrequency (RF) and/or other energy to target tissue to be ablated by system 10 (e.g., RF energy and/or other energy configured to electroporate tissue and/or otherwise ablate tissue). Alternatively or additionally, tip electrode 231 can be configured to deliver alternate tissue treatment (e.g., alternate ablative therapy) to target tissue, such as cryotherapy, ultrasonic therapy, and/or other ablative energies or modalities. Ablation catheter 200 can further include one, two, or more sensors and/or therapeutic elements (e.g., electrodes) positioned along shaft 210 (e.g., along a distal portion of shaft 210), ring electrode 232 shown. A first ring electrode 232 can be positioned proximate tip electrode 231, as shown. In some embodiments, catheter 200 comprises functional element 239 (e.g., a functional element comprising one, two, or more sensors, and/or one, two or more transducers), such as a functional element 239 positioned on the distal portion of shaft 210. In some embodiments, any electrode and/or other functional element of ablation catheter 200 can be configured to deliver therapy (e.g., ablative therapy) to tissue to be treated. In some embodiments, functional element 239 comprises one, two, or more electrode-based localization elements, such as conductive elements used to perform impedance-based localization. Alternatively or additionally, functional element 239 can comprise one, two, or more coils and/or other magnetic elements (“coils” herein), such as elements used to perform magnetic-based localization. In some embodiments, one or more functional elements 239 each comprise at least one electrode and at least one coil.

Ablation catheter 200 can include force assembly 240 which can be configured to provide signals related to the level of contact between a portion of ablation catheter 200 and tissue. Force assembly 240 can be positioned on and/or be integral to a distal portion of shaft 210. Force assembly 240 can be configured to measure (e.g., provide console 100 with information relating to) the force between tip electrode 231 and tissue, such as during an “ablation” (e.g., a delivery of energy at a single tissue location intended to provide electrical block and/or other therapeutic benefit to the patient). Additionally or alternatively, force assembly 240 can be configured to maintain, and/or to help maintain, a constant force between tip electrode 231 and tissue. Force assembly 240 can be of similar construction and arrangement to the similar components described in applicants co-pending U.S. patent application Ser. No. 16/335,893, titled “Ablation System with Force Control”, filed Mar. 22, 2019.

Force maintenance assembly 240 can be positioned within handle 220, within a portion of shaft 210 (e.g., within the distal portion of ablation catheter 200), and/or on the distal end of shaft 210. Force maintenance assembly 240 can comprise one, two, or more force maintenance elements, elements 245 shown. As examples, such force maintenance elements 245 can be and/or can include one or more of: a hydraulic element, a spring, a magnet, a compressible fluid, a memory material, and the like. Force maintenance elements 245 can be located at a distal end, proximal end, or intermediate portion of ablation catheter 200, or a combination of two or more of these. Force maintenance assembly 240 can also comprise one, two, or more sensing elements which can take the form of and/or include one or more sensors.

Force maintenance assembly 240 can be axially aligned with shaft 210 (e.g., a major axis of force maintenance assembly 240 is aligned with a central axis of the distal portion of shaft 210), such as when assembly 240 is aligned with the distal portion of shaft 210. Force maintenance assembly 240 can be configured to absorb mechanical shocks and/or it can be configured to dynamically (e.g., dynamically and automatically) respond to movement of the heart wall or other cardiac tissue (e.g., avoiding reliance on the clinician to manually react to the movement of the endocardial surface in a cardiac ablation procedure). Force maintenance assembly 240 can allow for high and/or low frequency movements, various movement ranges, etc. Force maintenance assembly 240 can be configured to compress over a “travel distance” (also referred to as the “compression distance” and is equal to the distance force maintenance assembly 240 compresses when a force is applied) up to a pre-determined maximum distance (the “max compression distance” or “max travel distance”), such as a maximum distance between 0.1 mm to 10 mm, such as a maximum distance between 0.1 mm and 5 mm, and/or some other predetermined distance range and/or limit. Force maintenance assembly 240 can be configured to be adjusted and/or calibrated, such as an adjustment and/or calibration of force maintenance assembly 240 that is performed during the manufacture of force maintenance assembly 240 and/or ablation catheter 100, and/or an adjustment and/or calibration that is performed in the clinical setting in which system 10 is used.

Force maintenance assembly 240 can be configured to provide a pre-determined force range over all or a portion of the travel distance, for example, a pre-determined constant and/or variable force between 0.1 gmf and 100 gmf, such as between 5 gmf and 30 gmf, and/or such as between 10 gmf and 30 gmf. In some embodiments, force maintenance assembly 240 is configured to provide a relatively constant force over all or at least a portion of the travel distance, for example, a pre-determined constant force between 0.1 gmf and 100 gmf, such as between 5 gmf and 30 gmf, or such as between 10 gmf and 30 gmf. Additionally or alternatively, in some embodiments, force maintenance assembly 240 is configured to provide a variable force over all or a portion of the travel distance, such as a variable force that varies within a pre-determined range of forces (e.g., a range of forces proportional to the amount compressed). For example, force maintenance assembly 240 can be configured to apply a force that varies between 5 gmf and 30 gmf, such as a force that varies between 10 gmf and 30 gmf. In some embodiments, the force applied by force maintenance assembly 240 is adjustable, such as to be adjusted by a clinician prior to and/or during a clinical procedure in which system 10 is used.

Force maintenance assembly 240 can include one, two, or more sensing elements or sensors configured to produce a signal correlating to the amount of compression of force maintenance assembly 240. Additionally or alternatively, the sensors can be configured to produce a signal correlating to compression (e.g., maximum compression) of force maintenance assembly 240.

Ablation catheter 200 can be configured for ablation and/or other treatment (“ablation” herein) of tissue of one or both atria of the heart (e.g., to create one or more lesions to treat atrial fibrillation and/or right atrial flutter) and/or ablation of tissue of the ventricles of the heart (e.g., to treat ventricular tachycardia). For ablation of the atria, force maintenance assembly 240 can be configured with a first max compression distance, such as a distance less than or equal to 10 mm, such as less than or equal to 5 mm, or such as less than or equal to 3 mm. Alternatively, for ablation of the ventricles, force maintenance assembly 240 can be configured with a second max compression distance, such as a distance greater than the first max compression distance, such as a distance at least 1 mm greater than the first max compression distance, such as a second (ventricular) max compression distance of at least 3 mm or at least 6 mm. In some embodiments, the first (atrial) max compression distance comprises a distance of approximately 2-3 mm. In some embodiments, the second (ventricular) max compression distance comprises a distance of approximately 4-6 mm.

In some embodiments, handle 220 of ablation catheter 200 comprises one or more functional elements, functional element 229. Functional element 229 can comprise one, two, or more elements (e.g., one, two or more sensors) configured to produce information that can be used to inform the user (e.g., an electrophysiologist or other clinician performing the clinical procedure) of an operational condition of system 10. In some embodiments, functional element 229 is configured to produce information related to an operational condition of ablation catheter 200, for example to inform the user when tip electrode 231 is in contact with the cardiac wall and/or to inform the user of the contact force being exerted on the cardiac wall by tip electrode 231. In some embodiments, handle 220 comprises a translucent housing, and functional element 229 comprises a light emitting element configured to illuminate the translucent housing to indicate the operational condition to the user. In some embodiments, functional element 229 comprises a haptic and/or an audible transducer configured to alert the user via haptic (e.g., vibrational) and/or audible signals. In some embodiments, console 100 activates functional element 229 (e.g., turns on and/or monitors signals received from functional element 229) when system 10 is in a particular operational state (e.g., an energy delivery state in which console 100 is configured to evaluate and/or otherwise monitor contact force and/or another condition related to energy delivery).

Mapping Catheter 300 can comprise shaft 310, with handle 320 positioned on the proximal end of shaft 310, and an array positioned on the distal portion of shaft 310. In some embodiments, the array comprises a basket array, basket 330. Basket 330 can comprise a plurality of splines, with each spline comprising one, two, or more functional elements configured to enable physical and/or electrical anatomic mapping of a body structure of the patient (e.g., a cardiac chamber of the patient). The functional elements of basket 330 can include one, two, or more electrodes, electrode 331 shown, one, two, or more ultrasound transducers, UST 332 shown, one, two, or more magnetic elements, coil 333 shown, and/or one, two, or more other functional elements, functional element 339 shown. Functional element 339 can comprise: one, two, or more sensors (e.g., electrodes, ultrasound sensors, pressure sensors, impedance sensors, and/or physiologic sensors); one, two or more transducers (e.g., heating elements, cooling elements, and/or other energy delivery elements); and/or one, two, or more other functional elements.

Mapping catheter 300 can be of similar construction and arrangement to the similar components described in applicants co-pending U.S. patent application Ser. No. 16/097,959, titled “Cardiac Mapping System with Efficiency Algorithm,” filed Oct. 31, 2018, and/or applicants co-pending U.S. Provisional Patent Application Ser. No. 62/939,412, titled “Tissue Treatment Systems, Devices, and Methods,” filed Nov. 22, 2019.

Auxiliary catheter 400 can comprise a diagnostic catheter, a mapping catheter, a treatment catheter, or a combination of two or more of these. Catheter 400 can comprise shaft 410, with handle 420 positioned on the proximal end of shaft 410, and array 430 positioned on the distal portion of shaft 410. Array 430 can comprise at least one functional element, functional element 439 (e.g., a functional element comprising one, two, or more sensors, and/or one, two or more transducers). For example, functional element 439 can comprise a plurality of electrodes. In some embodiments, catheter 400 comprises a lasso catheter (e.g., a mapping catheter biased in a looped configuration, as shown).

Sheath 70 can comprise shaft 71, with handle 72 positioned on the proximal end of shaft 71. At least one lumen, lumen 74, extends through at least a portion of the length of shaft 71 (e.g., from the proximal end of handle 72 to the distal end of shaft 71). Sheath 70 can comprise one, two, or more functional elements, functional element 79 shown. Functional elements 79 can comprise two or more similar or dissimilar elements (e.g., similar and/or dissimilar sensors, transducers, and/or other functional elements). In some embodiments, functional element 79 comprises one or more electrode-based localization elements, such as elements used to perform impedance-based localization. Alternatively or additionally, functional element 79 can comprise one or more coils and/or other magnetic elements (“coils” herein), such as elements used to perform magnetic-based localization. In some embodiments, functional element 79 comprises at least one electrode and at least one coil.

One or more devices of system 10 (e.g., ablation catheter 200 and/or mapping catheter 300) can be introduced into the patient via insertion through lumen 74 of sheath 70 (e.g., when at least the distal portion of sheath 70 has been inserted into the patient and the distal end is located proximate a procedural site, such as within a cardiac chamber of the patient). Sheath 70 can be of similar construction and arrangement to the similar components described in applicants co-pending International PCT Patent Application Serial Number PCT/US2019/031131, titled “Cardiac Information Processing System,” filed May 7, 2019.

Patch 60 can comprise one, two, or more standard skin electrodes and/or other electrodes configured to attach to the skin of the patient and transmit electrical signals through the patient and/or receive electrical signals from the patient. In some embodiments, patch electrodes 60 are configured to record the patient's heart electrical signals to generate an electrocardiogram (ECG) and/or to transmit and/or receive localization signals of system 10. Patch electrodes 60 can be configured to operably attach (e.g., electrically attach) to console 100.

Console 100 is configured to operably attach to one, two, or more components of system 10, such as one, two, or more patient insertable devices of system 10. Console 100 comprises one, two, or more wires, filaments, and/or other conduits, conduit 101, and one, two, or more attached connectors, connector 102. Each connector 102 can operably attach to a mating connector of a component of system 10 (e.g., a patient insertable device of system 10). Conduit 101 can comprise one or more wires or conductive traces (“wires” herein), optical fibers, tubes (e.g., hydraulic, pneumatic, irrigation or other fluid delivery tubes), wave guides, and/or mechanical linkages (e.g., translating filament), each of which can be used to operably attach one, two, or more components of the device to one, two, or more components of console 100. Console 100 can include an interface for connecting the devices of system 10 to the electronics within console 100, patient interface 105. Patient interface 105 can comprise circuitry configured to protect the patient from unintended energy delivery (e.g., shock) from the electronics within console 100. Additionally, patient interface 105 can comprise circuitry configured to protect console 100 from adverse effects that could otherwise result when an electrical energy (e.g., a cardioversion pulse, a pacing pulse, a defibrillation pulse, and/or ablation energy) is delivered to the patient (e.g., by a system 10 component or other energy delivery device). For example, patient interface 105 can comprise an electrical protection circuit configured to protect console 100 from damage caused by these high-energy signals electromagnetically coupling into one or more electrical conduits of system 10. Patient interface 105 can include one or more components selected from the group consisting of: a filter; a transformer; a buffer; an amplifier; a pass thru (e.g., a conduit that is unfiltered or otherwise unaltered by patient interface 105, such as a fluid conduit); and combinations of two or more of these.

Console 100 includes processing unit 110. Processing unit 110 can comprise at least one microprocessor, computer, and/or another electronic controller, processor 111. Processing unit 110 can also include one, two, or more algorithms, algorithm 115. Processor 111, via algorithm 115, can perform one or more of the processes described herein, such as a process performed in response to one or more commands the user inputs into system 10 via one or more graphical user interfaces, GUI 125 described herein. Processing unit 110 can comprise a memory module for storing instructions for performing algorithm 115, and the memory module can be electrically connected to processor 111. Processing unit 110 can receive a signal, such as a signal from one, two, or more functional elements of catheters 200, 300, and/or 400 (e.g., a signal from one, two, or more sensor-based functional elements of these devices). Processing unit 110 can be configured to perform one or more mathematical operations based on the received signal, and to produce a result correlating to a physiologic parameter of the patient and/or an operational parameter relating to at least one device of system 10. For example, the result can correlate to a quantitative and/or qualitative measure of the force applied by ablation catheter 200 to tissue, the orientation of ablation catheter 200 relative to tissue, the proximity of a portion of ablation catheter 200 to cardiac tissue, and/or the level or quality of contact between a portion of ablation catheter 200 (e.g., tip electrode 231) and cardiac tissue. The one or more mathematical operations can comprise an operation of function selected from the group consisting of: arithmetic operations; statistical operations; linear and/or non-linear functions; operations as a function of time; operations as a function of space or distance; comparison to a threshold; comparison to a range; and combinations of two or more of these. In some embodiments, algorithm 115 is configured to determine and/or assess at least one of contact, force, or pressure applied by ablation catheter 200 to tissue. In some embodiments, algorithm 115 processes one or more signals received from one or more sensors of system 10 (e.g., one or more functional elements of system 10 comprising a sensor), such as a signal correlating to: the temperature of the energy delivery element; the temperature of the tissue surrounding the energy delivery element; the duration of energy delivery to tissue; the level of energy being delivered to tissue; the force and/or pressure being applied to tissue; and combinations of two or more of these. In some embodiments, algorithm 115 is configured to control a parameter of a treatment procedure (e.g., the level of ablative energy delivered to tissue) in a closed loop fashion. Processing unit 110, processor 111, and/or algorithm 115 can be of similar construction and arrangement to the similar components described in applicant's co-pending U.S. patent application Ser. No. 16/335,893, titled “Ablation System with Force Control,” filed Mar. 22, 2019, applicant's co-pending international PCT Patent Application Serial Number PCT/US2020/028779, titled “System for Creating a Composite Map,” filed Apr. 17, 2020, and/or applicant's co-pending International PCT Patent Application Serial Number PCT/US2019/031131, titled “Cardiac Information Processing System”, filed May 7, 2019.

Console 100 can include an interface for providing and/or receiving information to and/or from a user of system 10, user interface 120. User interface 120 can include one, two, or more user input and/or user output components. For example, user interface unit 120 can comprise a joystick, keyboard, mouse, touchscreen, and/or other human interface device, user input device 121 shown. In some embodiments, user interface unit 120 comprises a display, such as display 122, also shown. Processor 110 can provide a graphical user interface, GUI 125, to be presented on display 122.

Console 100 can include localization module 130. Localization module 130 can transmit energy and/or signals to ablation catheter 200, mapping catheter 300, and/or patches 60 via patient interface 105 (as shown), or otherwise. Localization module 130 can be configured to transmit one, two, or more signals into the patient (e.g., via one, two, or more patches 60), such as to create a localization field within the patient. Furthermore, localization module 130 can receive signals from one, two, or more electrodes (or other sensors) of ablation catheter 200 and/or mapping catheter 300, such as signals correlating to the localization signals, such as to determine the localization of the one or more electrodes within the localization field (e.g., to determine the location and/or orientation of the associated catheter(s) within the patient, “localize” herein). In some embodiments, two or more localization fields can be used simultaneously. The components used to generate and/or sense the localization fields (e.g., patches 60 and/or the one or more electrodes of ablation catheter 200 or mapping catheter 300), can be configured to transmit localization signals (herein “source”), receive localization signals (herein “sink”), and/or transmit and receive localization signals interchangeably. For example, the components can be multiplexed to source and sink localization signals between each other in a pattern configured to enhance the localization information received by localization module 130, such as information regarding the relative position between a component of system 10 and the cardiac tissue or other structures within the cardiac chamber and/or another component of system 10.

Console 100 can include mapping module 1410. Mapping module 140 can be configured to record and/or process (e.g., process via processing unit 110) information collected from one, two, or more patient insertable devices of system 10. Mapping module 140 can be configured to process the recorded data to produce one, two, or more maps of anatomical and/or electrophysiological information of the patient. Mapping module 140 can record and/or process ultrasound information (e.g., information recorded from one or more UST 332 of mapping catheter 300), and/or biopotential information (e.g., information recorded from one or more electrodes, such as one or more electrodes 331 of mapping catheter 300).

Mapping nodule 140 can transmit energy and/or signals to ablation catheter 200, mapping catheter 300, and/or patches 60, such as via patient interface 105 (as shown), or otherwise. Mapping module 140 can be configured to transmit one, two, or more signals into the patient (e.g., via one or more patches 60), such as to create a localization field within the patient. Furthermore, mapping module 140 can receive signals from one or more electrodes (or other sensors) of ablation catheter 200 and/or mapping catheter 300, such as signals correlating to the localization signals, such as to determine the location of the one or more electrodes within the localization field (e.g., to determine the location and/or orientation of the associated catheter(s) within the patient). In some embodiments, two or more localization fields can be used simultaneously. The components used to generate and/or sense the localization fields (e.g., patches 60 and/or the one or more electrodes of ablation catheter 200 or mapping catheter 300), can be configured to transmit localization signals (herein “source”), receive localization signals (herein “sink”), and/or transmit and receive localization signals interchangeably. For example, the components can be multiplexed to source and sink localization signals between each other in a pattern configured to enhance the localization information received by mapping module 140, such as information regarding the relative position between a component of system 10 and the cardiac tissue or other structures within the cardiac chamber and/or another component of system 10.

Mappings module 140 can be configured to produce one, two, or more maps of biopotential activity (e.g., electrical activity of a cardiac chamber over a period of time) to be displayed to the user via GUI 125. The one or more maps can be similar to the one or more maps described in applicant's co-pending U.S. patent application Ser. No. 16/097,955, titled “Cardiac information Dynamic Display System and Method”, filed Oct. 31, 2018.

Console 100 can include imaging module 150. Imaging module 150 can be configured to transmit and receive ultrasound signals via one, two, or more UST 332 of mapping catheter 300 to determine the distance between each UST 332 and the cardiac tissue, such as to, in coordination with the localization data, generate an anatomical model of the cardiac tissue. Additionally or alternatively, imaging module 150 can be configured to import image data from an external imaging device, such as a fluoroscopy device, an MRI machine, and/or other medical imaging devices. In some embodiments, imaging module 150 is configured to register two or more anatomic models of cardiac tissue, such as a model generated using the ultrasound capabilities of mapping catheter 300 and an imported anatomy from an MRI. In some embodiments, imaging module 150 is of similar construction and arrangement to the similar components described in applicant's co-pending U.S. patent application Ser. No. 15/569,185, titled “Ultrasound Sequencing System and Method”, filed Oct. 25, 2017.

Console 100 can include therapy module 160. Therapy module 160 can include a unit configured to deliver ablative energy to tissue, energy delivery unit 161. For example, energy delivery unit 161 can deliver RF or other ablative energy to tissue via tip electrode 231 of ablation catheter 200. In some embodiments, energy delivery unit 161 comprises a pass through unit configured to receive energy from an external source (e.g., an ablation generator operably connected to console 100), such as to deliver the received energy to ablation catheter 200 or other component of system 10. Therapy module 160 can include force module 162. Force module 162 can be configured to determine the contact force between a portion of ablation catheter 200 (e.g., tip electrode 231) and tissue. Force module 162 can receive signals from force assembly 240 and analyze these signals (e.g., analyze via processing unit 110) to determine the contact force. In some embodiments, force assembly 240 comprises an assembly configured to manipulate the position of tip electrode 231 relative to tissue, such as to maintain a constant force between tip electrode 231 and the tissue. In these embodiments, force module 162 can be configured to instruct and/or provide motive force to cause assembly 240 to maintain a desired force. For example, force module 162 can provide pressurized fluid to force assembly 240 comprising a piston assembly configured to provide a constant force over the travel distance of the piston assembly. In some embodiments, force assembly 240 and/or force module 162 are of similar construction and arrangement to the similar components described in applicants co-pending U.S. patent application Ser. No. 16/335,893, titled “Ablation System with Force Control”, filed Mar. 22, 2019.

In some embodiments, console 100 is configured to measure (e.g., predict) the efficacy of a tissue treatment (e.g., lesion creation) performed by ablation catheter 200 (e.g., a prediction of the 6 month and/or 12 month efficacy of a lesion created by applying RF and/or other energy to tissue via tip electrode 231 of ablation catheter 200). Such an efficacy measurement may be a function of one, two, or more ablation parameters (“input data” herein), such as ablation time, ablation power (e.g., average power delivered and/or cumulative power delivered), and/or contact force between the one or more ablative elements delivering energy (e.g., tip electrode 231) and the tissue receiving the energy. In some embodiments, contact force is measured by force module 162 as described herein. Algorithm 115 can be configured to calculate a value indicative of the efficacy (e.g., a predicted 6 and/or 12 month efficacy) of an ablation (e.g., an “ablation index”), lesion quality index 170. Lesion quality index 170 can represent the current status of a lesion being created (e.g., a value which changes as energy is being delivered), and/or the status of an already created lesion (e.g., provided in a table or other form for a single lesion or set of previously created lesions). Algorithm 115 can implement a transfer function to determine lesion quality index 170 from the input data. Algorithm 115 can apply the following dependencies in determining the value of lesion quality index 170 based on the input data: lesion quality index 170 is unbounded with respect to time and should equal zero when time t equals zero (e.g., at the initiation of the delivery of ablation energy); lesion quality index 170 should be unbounded with respect to power and should equal zero when the cumulative power delivered is equal to zero (e.g., at the initiation of the delivery of ablation energy); and/or the effect of contact force should be equivalent to that of a step function (e.g., a smoothed step function), such that an ablation is considered ineffective (e.g., predicted to be therapeutically ineffective) if the contact force (e.g., the average, minimum, and/or cumulative contact force that occurs during an ablation) is too small, and an ablation is considered effective if the contact force (e.g., the average, minimum, and/or cumulative contact force that occurs during an ablation) is greater than some minimum value, while increasing the contact force beyond a minimum value does not increase the effectiveness of the ablation.

In some embodiments, algorithm 115 performs the following calculation which satisfies the dependencies identified hereabove:

W(t,\hat{P},\hat{F})=κ+W_{∞} ln(αt+1)ln(β\hat{P}+1)(1−exp(−γ\hat{F}))
with

$$\hat{P} = {\int }_{{t}_{0}}^{T} P ( t ) dt \\ \hat{F} = \frac{1}{( T - {t}_{0} )} {\int }_{{t}_{0}}^{T} F ( t ) dt or \hat{F} = {\int }_{{t}_{0}}^{T} F ( t ) dt$$

where t₀ is time at the start of ablation, following stabilization of the location of the ablation catheter tip while in contact with the endocardial wall (e.g., once tip electrode 231 has made contact with the endocardial wall and the position of the electrode is relatively fixed relative to the wall). For example, the location of the ablation catheter tip can be considered stable while the motion of the catheter tip is maintained within a radius of no more than 10 mm, such as no more than 5 mm, such as no more than 2 mm, for a minimum of at least 1 sec, such as at least 3 sec. In the equation shown hereabove, T is the time at the end of an ablation. Power is left non-normalized in order to retain the energy equivalence of different (P,T) combinations. Force may be expressed as either a normalized integral or it can remain in non-normalized form.

Using an appropriate data set consisting of multiple measurements of lesion size versus the input data (e.g., ablation time, power, and contact force), the 5 parameters κ, W_{∞}, α, β, γ can be obtained by algorithm 115 using an iterated non-linear least squares technique. Once fitted to a representative data set, the resulting constants define a transformation from real-time measured quantities to a prediction of lesion quality (e.g., a value for lesion quality index 170). System 10 can provide the value produced by the transformation (e.g., lesion quality index 170) via GUI 125 without dimensions. In some embodiments, the value produced by the transformation performed by algorithm 115 can be subsequently scaled (e.g., via a constant of 10 or greater) by algorithm 115, such that the resultant lesion quality index 170 value will not be confused with a value representing a prediction of the size of a lesion (e.g., the length of a lesion). In other words, a scaling factor can be used to avoid potential index values under 20, such as to avoid confusion with lesions up to 20 mm in length. For example, a non-scaled value of 2.5 may be interpreted as a predicted lesion size of 2.5 mm, while a scaled value of 75 (e.g., using a scale value of 30), is less likely to be interpreted as a predicted lesion size.

System 10 can be configured as a force-sensing ablation system that produces contact data that includes a bias (e.g., in the determined force) which is a function of contact angle (e.g., is related to contact angle). If this bias is known, contact force may be scaled by contact angle as, for example

\hat{F}ₛ=\hat{F}(C₁ cos²(θ)+C₂ sin²(θ))

where θ is the contact angle relative to the surface and the two constants C₁ and C₂ are determined empirically by evaluation of the variation of contact force in a controlled (e.g., in vitro) environment.

In some embodiments, lesion quality index 170 is provided (e.g., displayed or otherwise provided) to the user via GUI 125 during the time at which an ablation is being performed, such as in a real time and/or other relatively continuous fashion in which index 170 is continuously updated to reflect the current status of the lesion being created. The continuous updating of index 170 can be provided such that monitoring can be performed (e.g., by system 10 and/or a user of system 10) to determine when delivery of energy should be stopped (e.g., manually by a user of system 10 and/or automatically by system 10). In some embodiments, system 10 is configured to indicate the successful creation of a lesion when index 170 reaches a first level (e.g., a level at which a user of system 10 can manually stop energy delivery), and if energy delivery is continued (e.g., not stopped by the clinician), system 10 automatically stops the energy delivery (e.g., when index 170 reaches a second level, greater than the first level). In some embodiments, GUI 125 provides lesion quality index 170 (e.g., provides the current value of index 170) to the user in the form of visual information, audible information, and/or tactile information.

In some embodiments, GUI 125 provides lesion quality index 170 to the user in the form of visual information comprising alphanumeric information representing index 170 (e.g., a numeric scale and/or text similar to “partially completed”, “fully completed”, and the like).

In some embodiments, GUI 125 provides lesion quality index 170 to the user in the form of visual information comprising graphical information representing index 170 (e.g., a bar graph, pie chart, changing color scheme, and the like).

In some embodiments, GUI 125 provides lesion quality index 170 to the user in the form of audible information, such as when provided by a speaker delivering a sound that changes (e.g., changes in frequency, output, and/or other audio property) as index 170 changes.

In some embodiments, GUI 125 provides lesion quality index 170 to the user in the form of haptic information, such as when provided using a vibrational transducer of ablation catheter 200 (e.g., via functional element 229 comprising a vibrational transducer) whose vibration amplitude and/or frequency changes as index 170 changes.

In some embodiments, GUI 125 provides lesion quality index 170 to the user in the form of visual information (e.g., as described herein), and at least one of audible information and/or haptic information.

During an ablation of the present inventive concepts, there are many physical orientations in which ablation catheter 200 may effectively be used to supply energy (e.g., RF energy and/or other energy) to tissue to effectively ablate the tissue. The detection (e.g., quantitative and/or qualitative assessment and/or other detection by console 100) of all relevant interactions between catheter 200 and the tissue proximate catheter 200 (e.g., orientations, contact force, and/or other such interactions, “tissue interactions” herein) for efficacious lesion creation is difficult to perform (e.g., to analytically determine). A large subset of tissue interaction conditions may be analytically modelled, but these may not be the more common conditions incurred in the procedure.

In some embodiments, console 100 is configured to employ data classification methods to aid in determining a current tissue interaction set of conditions (e.g., in determining a level of contact between tip electrode 231 and tissue). For example, using an impedance detection system (e.g., a detection system based on the localization methods as described herein), the tip electrode 231 and ring electrode(s) 232 impedances can be monitored, such as via monitoring performed at a specific frequency. Impedances can be measured as voltages that are induced as a result of providing a differential current between two ring electrodes 232. The data collected represents different conditions when at least a portion of ablation catheter 200 is or is not in contact with tissue (e.g., is or is not in a minimum level of contact with tissue). In some embodiments, tissue contact can be determined by system 10, and correlation between the collected data and the determined contact can be performed.

In some embodiments, data classification can be performed by system 10 by correlating individual data sets in N-dimensions that show specificity to a desired outcome, such as specificity in determining contact between catheter 200 and tissue. In some embodiments, two or three-dimensional graphical plots can be used by system 10 to demonstrate the specificity of the classification, for example plots displayed to the user on GUI 125.

In some embodiments, individual data streams are classified in the frequency domain. Using frequency domain analysis, energy in different frequency bands on ring electrodes 232 can be used to determine catheter/tissue contact. This technique is an analogue of bandwidth filtering in the time domain. In the frequency domain, all frequencies in a data stream can be monitored simultaneously thereby facilitating their correlative properties. Fast Fourier transform (FFT) correlation can be performed (e.g., in real time) to aid the user during ablation procedures. Additionally or alternatively, system 10 can be configured to perform time domain correlation between recorded signals (differentially) as a method of classification. This time domain correlation configuration has the advantage of removing common mode signals (common mode correlation) and mitigating noise issues.

In some embodiments, console 100 is configured to determine a contact state (e.g., contact force, contact angle, and/or other contact parameter) between ablation catheter 200 and a portion of the cardiac wall. Console 100 (e.g., algorithm 115) can be configured to determine the contact state by implementing k-Mean clustering of measurements made in 4D space. As an example, a state vector (V₁,V₂,V₃,V₄) can be defined in [symbol] ⁴-space (e.g., 4-dimensional voltage space) as:

P∈ [symbol] ⁴=(V_{Tip},V_{Band},V_{Source},V_{Sink})

where P defines a point in four-dimensional space. Electric field distortion caused by different contact states of ablation catheter 200 can change the location (coordinates) of point P in [symbol] ⁴-space. The contact state can be a set of labels C={C₁, C₂, . . . , Cₖ} which can be defined as:

C={no-contact, lateral contact, axial contact, force maintaining contact, cantilever, . . . }.

Using k-mean clustering, partitions among different contact states can be found by minimizing the relation

$${min}_{C} {\sum }_{i = 1}^{k} {\sum }_{P ∈ {C}_{i}} {‖ P - {μ}_{i} ‖}^{2} ,$$

where μᵢ is the mean of points in Cᵢ. Knowing the partitions between different contact states Cᵢ in [symbol] ⁴-space, any later measurement P′=(V′_{Tip},V′_{Band},V′_{Source},V′_{Sink}) can be classified by calculating its Euclidian distance from the mean of each class μᵢ. In this configuration, each contact state, Cᵢ, occupies a certain region in [symbol] ⁴-space. In other words, the location of P′ with respect to the mean of each class μᵢ can be used (e.g., used by algorithm 115) to determine the contact state of ablation catheter 200.

In some embodiments, system 10 is configured to determine the distance between an electrode (e.g., an electrode of ablation catheter 200 as described herein) and the endocardial surface of a heart chamber (e.g., the “proximity” of the electrode to the surface) by measuring changes in an electrical field (e.g., an impedance-field) proximate the electrode. In some embodiments, a body-surface patch-electrode (e.g., patch 60) is used as an indifferent reference against which the impedance-field measurements are made and from Which all 3D localization values can be derived. Similarly, pairs of patch-electrodes can be positioned around the torso of the patient and currents can be driven between the pairs of electrodes to establish an impedance-field (e.g., a broad, unipolar 3D impedance-field). Empirical experience with this impedance-field approach revealed that the quadrature (“imaginary”) component of demodulated impedance signals are generally sensitive to distance from the interface between two differing biologic materials, for example between the blood in a heart chamber and its associated endocardial surface tissue. In contrast, the in-phase (“real”) component of demodulated impedance signals are generally sensitive to the raw, physical location of an electrode at any point within the tissues. This real component includes a more limited sensitivity to the boundaries of tissue interfaces than is observed with the quadrature component. In some embodiments, the impedance-field and the measured signals are applied broadly, across the whole torso, in an electrically unipolar configuration (“configuration 1” herein). Configuration 1 results in a relatively small signal-to-noise ratio and a lower level of sensitivity to both location and to proximity to tissue interfaces (e.g., a smaller signal-to-noise ratio and a lower level of sensitivity results than those achieved using a locally driven impedance-field, “configuration 2” described herebelow). In addition, configuration 1 results in derived electrode locations and proximity measurements to the endocardial surface are correspondingly limited in specificity, accuracy, and variability, than those achieved using configuration 2 (i.e. a locally driven impedance-field).

As described hereabove, system 10 can overcome the limitations of configuration 1 (e.g., limitations in specificity, accuracy, and variability) by applying an alternate methodology in both impedance-field drive and in the derivation of electrode-proximity (i.e. configuration 2). For example, the impedance-field can be driven locally, between two or more electrodes on the catheter (e.g., ablation catheter 200). This approach significantly increases the sensitivity to a tissue interface, as the abrupt change in the dielectric values at the boundary markedly distort the locally-driven, bipolar impedance-field of configuration 2. The components of magnitude and phase of the measured signal are correspondingly sensitive to the localized distortion in the driven impedance-field. The total, complex expression (e.g., the magnitude and phase) of the measured impedance-signal can be applied in a biophysical model that is based on electrostatic field-theory. This model incorporates fundamental electrical conditions that exist at the boundary of differing tissue (e.g., boundary-conditions between differing dielectrics, for example blood and epicardial tissue). The resultant values of proximity measurements obtained from this unique methodology (configuration 2) are significantly more specific, accurate, and less variable than the broadly unipolar method (configuration 1).

In some embodiments, the parameters used to determine the lesion quality index 170 are displayed to an operator (e.g., a clinician) via user interface 120. In some embodiments, a clinician can vary one or more parameters to determine the impact (e.g., the quantitative impact) of changing one or more of the parameters (e.g., in a subsequent tissue treatment to be performed). In some embodiments, algorithm 115 can determine a recommended level of one or more of the parameters (e.g., prescribe treatment) for a current lesion (e.g., a lesion currently being created) and/or for a lesion to be created in the future. In some embodiments, algorithm 115 comprises a machine learning, neural network, and/or other artificial intelligence algorithm (“AI algorithm” herein) that is configured to be trained by data collected from lesion creation for a current patient and/or patients previously treated by system 10 (e.g., the current system 10 and/or other systems 10 at other clinical facilities). This AI algorithm-based algorithm 115 can then provide feedback to an operator prior to, during, and/or after creation of one or more lesions in a patient.

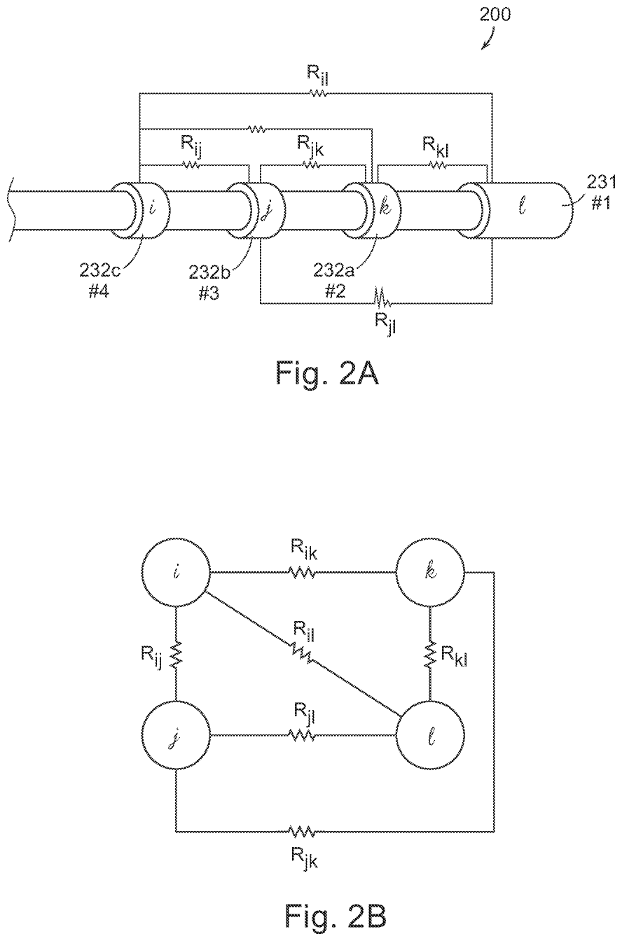

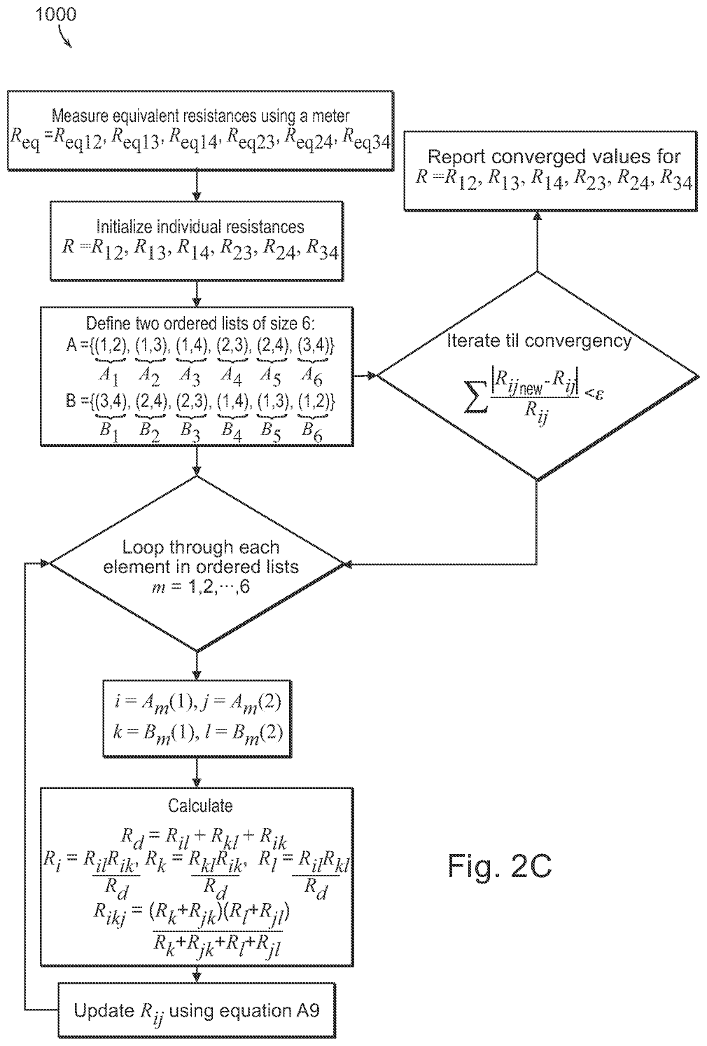

Referring now to FIGS. 2A and 2B, a perspective view of the distal portion of an ablation catheter, and a schematic view of a resistor network model are illustrated, respectively, consistent with the present inventive concepts. In some embodiments, console 100 is configured to determine the proximity and/or orientation of the distal portion of ablation catheter 200 relative to tissue, such as relative to the wall of a cardiac chamber when the distal portion of ablation catheter 200 is positioned within the cardiac chamber. For example, by determining the resistance between pairs of electrodes of ablation catheter 200, console 100 can estimate the electrical properties of the tissue surrounding these electrodes (e.g., blood or cardiac wall tissue), and using electrical properties information, the position and/or orientation of catheter 200 relative to tissue can be estimated by console 100. When positioned within a conductive medium (e.g., blood), the multiple electrodes of ablation catheter 200 can form an electrical circuit. At certain frequencies, for example frequencies above 20 kHz, this electrical circuit can be modeled as a resistor network, such as is shown in FIG. 2B. This resistor network model can be used to build a series of non-linear equations that can be used by system 10 to solve for individual resistance values between any two electrodes of ablation catheter 200 (e.g., between any two of tip electrode 231 and/or ring electrodes 232a-c). The following equations can be used to determine an equivalent resistance between any two electrodes:

$${R}_{d} = {R}_{il} + {R}_{kl} + {R}_{ik} \qquad ( A1 )$$

$${R}_{i} = \frac{{R}_{il} {R}_{ik}}{{R}_{d}} = {f}_{1} ( {R}_{ik} , {R}_{il} , {R}_{kl} ) \qquad ( A2 )$$

$${R}_{k} = \frac{{R}_{kl} {R}_{ik}}{{R}_{d}} = {f}_{2} ( {R}_{ik} , {R}_{il} , {R}_{kl} ) \qquad ( A3 )$$

$${R}_{l} = \frac{{R}_{il} {R}_{kl}}{{R}_{d}} = {f}_{3} ( {R}_{ik} , {R}_{il} , {R}_{kl} ) \qquad ( A4 )$$

$${R}_{lkj} = \frac{( {R}_{k} + {R}_{jk} ) ( {R}_{l} + {R}_{jl} )}{{F}_{k} + {R}_{jk} + {R}_{l} + {R}_{jl}} = {f}_{4} ( {R}_{ij} , {R}_{ik} , {R}_{il} , {R}_{jk} , {R}_{jl} , {R}_{kl} ) \qquad ( A5 )$$

$${R}_{{eq}_{ij}} = \frac{{R}_{ij} ( {R}_{i} + {R}_{lkj} )}{{R}_{ij} + {R}_{i} + {R}_{lkj}} = {f}_{5} ( {R}_{ij} , {R}_{ik} , {R}_{il} , {R}_{jk} , {R}_{jl} , {R}_{kl} ) \qquad ( A6 )$$

In these equations above, i, j, k, and l can correlate to any electrode (an example is illustrated in FIG. 2). Resistances Rᵢⱼ, Rᵢₖ, Rᵢₗ, Rⱼₖ, Rⱼₗ, Rₖₗ are the resistance between any two electrodes. Equation A6 relates the equivalent resistance, which can be measured by system 10 to the individual resistances: Rᵢⱼ, Rᵢₖ, Rᵢₗ, Rⱼₖ, Rⱼₗ, Rₖₗ. By substituting for Rᵢ, Rₖ, Rₗ, Rₗₖⱼ in R_{eq}_{ᵢⱼ} equation A6 yields:

$${R}_{{eq}_{ij}} = \qquad ( A7 )$$

$$\frac{\begin{matrix}{R}_{ij} ( \frac{{R}_{il} {R}_{ik}}{{R}_{il} + {R}_{kl} + {R}_{ik}} + \\ \frac{( \frac{{R}_{kl} {R}_{ik}}{{R}_{il} + {R}_{kl} + {R}_{ik}} + {R}_{jk} ) ( \frac{{R}_{il} {R}_{kl}}{{R}_{il} + {R}_{kl} + {R}_{ik}} + {R}_{jl} )}{\frac{{R}_{kl} {R}_{ik}}{{R}_{il} + {R}_{kl} + {R}_{ik}} + {R}_{jk} + \frac{{R}_{il} {R}_{kl}}{{R}_{il} + {R}_{kl} + {R}_{ik}} + {R}_{jl}} )\end{matrix}}{\begin{matrix}{R}_{ij} + \frac{{R}_{il} {R}_{ik}}{{R}_{il} + {R}_{kl} + {R}_{ik}} + \\ \frac{( \frac{{R}_{kl} {R}_{ik}}{{R}_{il} + {R}_{kl} + {R}_{ik}} + {R}_{jk} ) ( \frac{{R}_{il} {R}_{kl}}{{R}_{il} + {R}_{kl} + {R}_{ik}} + {R}_{jl} )}{\frac{{R}_{kl} {R}_{ik}}{{R}_{il} + {R}_{kl} + {R}_{ik}} + {R}_{jk} + \frac{{R}_{il} {R}_{kl}}{{R}_{il} + {R}_{kl} + {R}_{ik}} + {R}_{jl}}\end{matrix}}$$

Equation A7 is a highly nonlinear function, and system 10 is configured to solve for the individual resistances based on the equivalent resistance between electrodes i and j.

Referring additionally now to FIG. 2C, a method of solving a highly nonlinear function is illustrated, consistent with the present inventive concepts. Method 1000 can be executed by console 100 (e.g., using algorithm 115 executed on processor 111) to solve for equation A7 to determine the resistance between two electrodes of ablation catheter 200. Expansion of equation A6 yields:

R_{eq}_{ᵢⱼ}Rᵢⱼ+R_{eq}_{ᵢⱼ}Rᵢ+R_{eq}_{ᵢⱼ}Rₗₖⱼ=Rᵢⱼ(Rᵢ+Rₗₖⱼ) (A8)

Solving for Rᵢⱼ results in:

$${R}_{ij} = \frac{{R}_{{eq}_{ij}} ( {R}_{i} + {R}_{ikj} )}{{R}_{i} + {R}_{lkj} - {R}_{{eq}_{ij}}} = f ( {R}_{ik} , {R}_{il} , {R}_{jk} , {R}_{jl} , {R}_{kl} ) \qquad ( A9 )$$

Console 100 can measure the equivalent resistance between each pair of electrodes (e.g., electrodes #1-#4 as shown) of ablation catheter 200, resulting in the following sets of electrode-pair measurements: #1-#2; #1-#3; #1-#4; #2-#3; #2-#4; #3-#4. Using these measurements and equation A6, console 100 can solve (e.g., via method 1000) for the unknown resistance values between each pair of electrodes. Method 1000 illustrates an iterative method of solving for the resistance between each pair of electrodes based on the measured equivalent resistances.

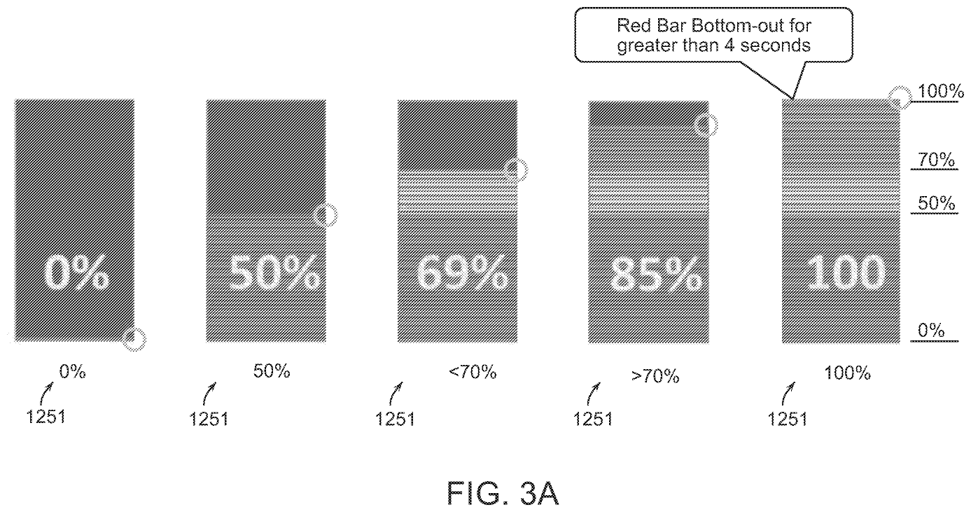

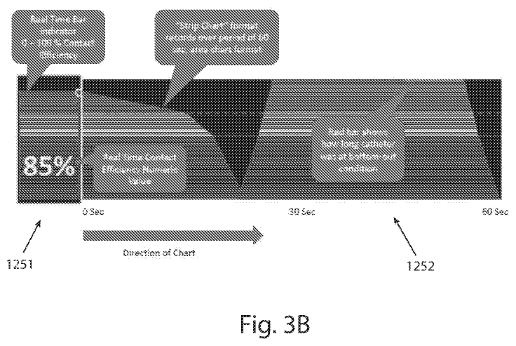

Referring now to FIGS. 3A and 3B, an indicator in a series of system states and a set of indicators showing instantaneous information and information over time are illustrated, respectively, consistent with the present inventive concepts. Console 100 can be configured to determine one or more characteristics of the contact state between tip electrode 231 of ablation catheter 200 and the cardiac wall, such as contact force, contact angle, and/or other contact parameter. In some embodiments, console 100 analyzes one or more contact parameters to determine the likely efficiency and/or effectiveness of an ablation performed with the determined contact, this efficiency referred to herein as the “contact efficiency”. In some embodiments, the contact efficiency can be determined based on the percentage of intended ablation time in which electrode 231 is in contact with tissue for a given ablation. In some embodiments, GUI 125 can include one or more displays indicating the contact efficiency to the user. FIG. 3A illustrates five examples of an efficiency indicator, indicator 1251 shown, representing a “real-time” graph of contact efficiency (e.g., the contact efficiency as determined by console 100 relating to the current position and/or contact force of tip 231 at the time the graph is displayed). Efficiency indicator 1251 can comprise a color-coded bar indicator, for example, an indicator that indicates the current efficiency, such as an indicator that displays a “good” color (e.g., green) when the efficiency is above a threshold (e.g., a 70% threshold), and a “bad” color or range of colors (e.g., not green) when the efficiency is below the threshold. For example, indicator 1251 can transition through a range of colors as the efficiency level changes, such as to indicate to a user that the efficiency resulting from manipulation catheter 200 (e.g., changing the tissue interaction) is approaching or departing from the threshold. In some embodiments, efficiency indicator 1251 is configured to indicate that a maximum measurable force condition has been reached, for example, when force assembly 240 comprises a piston assembly, and the piston is in a fully compressed state (e.g., higher levels of actual force do not result in a higher reading). In some embodiments, efficiency indicator 1251 is configured to indicate if the maximum measurable force condition is present for a time exceeding a threshold, for example, a threshold of two seconds, three seconds, and/or four seconds. In some embodiments, the data displayed via efficiency indicator 1251 can represent a running average of data, for example, an average of data over a five second window.

FIG. 3B illustrates a graph of contact efficiency over time, efficiency graph 1252. In some embodiments, efficiency graph 1252 comprises a graph representing data over a rolling window of time, for example, a 60 second window (e.g., the previous 60 seconds, with the left side portion of the graph representing current time). In FIG. 3B, efficiency indicator 1251 is illustrated adjacent to efficiency graph 1252, as the two may be displayed on GUI 125 simultaneously. In some embodiments, the length (e.g., length of time displayed) of graph 1252 can be at least the length of the longest planned ablation in a procedure (e.g., at least 60 seconds), such that during an ablation, graph 1252 displays the contact efficiency over the duration of the ablation. Similar to efficiency indicator 1251, efficiency graph 1252 can indicate a time period where a maximum measurable force condition was present (e.g., via a line or other maximum level indicator shown on graph 1252).

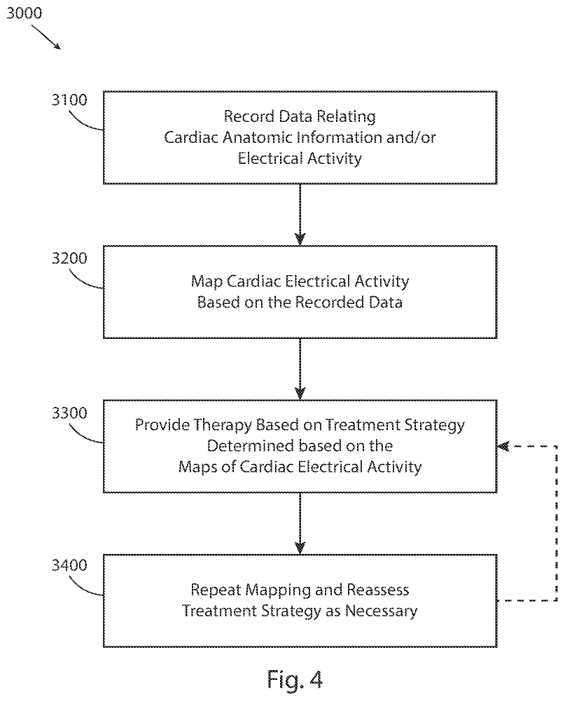

Referring now to FIG. 4, a method of providing a therapy is illustrated, consistent with the present inventive concepts. Method 3000 illustrates a process of providing a therapeutic strategy (e.g., a mapping and/or ablation strategy) to a user of system 10 to treat one or more cardiac conditions. In Step 3100, anatomic and/or electrical activity data is recorded by system 10, for example, data recorded using at least mapping catheter 300. In Step 3200, one or more maps of the cardiac electrical activity are produced by system 10 (e.g., one or more surface charge maps, dipole density maps, and/or voltage maps), and are further provided to the user via GUI 125. The recording and mapping of the cardiac electrical activity data can be performed using similar processes as those described in applicants co-pending U.S. patent application Ser. No. 16/097,955, titled “Cardiac Information Dynamic Display System and Method”, filed Oct. 31, 2018.

In Step 3300, based on the information provided via the one or more maps of cardiac activity, therapy (e.g., ablative therapy) can be provided to the patient. In some embodiments, a therapeutic strategy provided by system 10 (e.g., as determined by algorithm 115) can include a set of one or more energy delivery patterns collectively configured to address each conduction pattern identified (e.g., each aberrant pattern identified in the one or more maps). For example, a proposed first set of energy delivery patterns (e.g., a set of one or more energy delivery patterns) can comprise an energy delivery pattern that creates a set of lesions in a first pattern, such as a rosette pattern. The therapeutic strategy can further include a second set of energy delivery patterns, such as a set including a segment (e.g., a relatively short segment) connecting the first pattern to either an anatomical structure such as a valve ring and/or to another lesion set (e.g., an isolation line for one or more pulmonary veins). Alternatively or alternatively, a therapeutic strategy provided by system 10 can include providing a suggested pattern of energy delivery that transects the core of the identified conduction pattern whereby each end of the line of energy delivery is anchored in either an anatomical structure or previous energy delivery line (e.g., isolation line) for the pulmonary veins. In some embodiments, console 100 is configured to establish prevalence (e.g., prevalence of an aberrant conduction pattern) based on frequency of the conduction pattern. In some embodiments, a therapeutic strategy (e.g., as provided by algorithm 115) can include a bias (e.g., a threshold-based bias) towards treating aberrant conduction patterns that exhibit a frequency of activation of greater than or equal to once per second. Alternatively or additionally, a therapeutic strategy can include a bias towards treating an area where multiple activation patterns occur within a time period, for example a time period of approximately 4 seconds, 5 seconds, or 10 seconds.

In Step 3400, additional data is recorded using system 10, and the one or cardiac activity maps are updated, representing the cardiac electrical activity that is present after the treatment of Step 3300 (or mid treatment, for example, after one of several planned energy delivery patterns are delivered). In some embodiments, after the one or more maps are updated, the therapeutic strategy can be modified by system 10, and/or it can be confirmed to be “working as expected” (e.g., acceptable) and treatment can continue (e.g., continue per the previous strategy or per a modified strategy). Steps 3300 and 3400 can be repeated any number of times throughout a procedure, for example, until a desirable outcome of the treatment has been achieved.

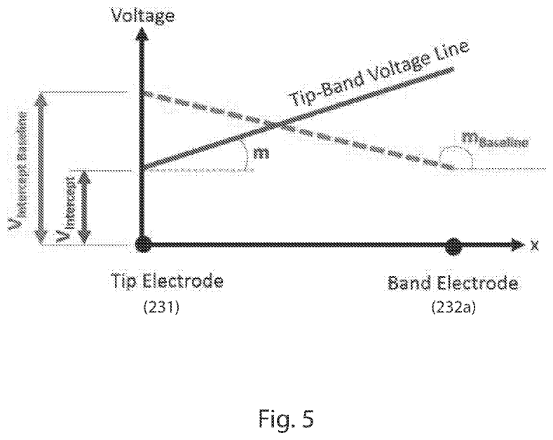

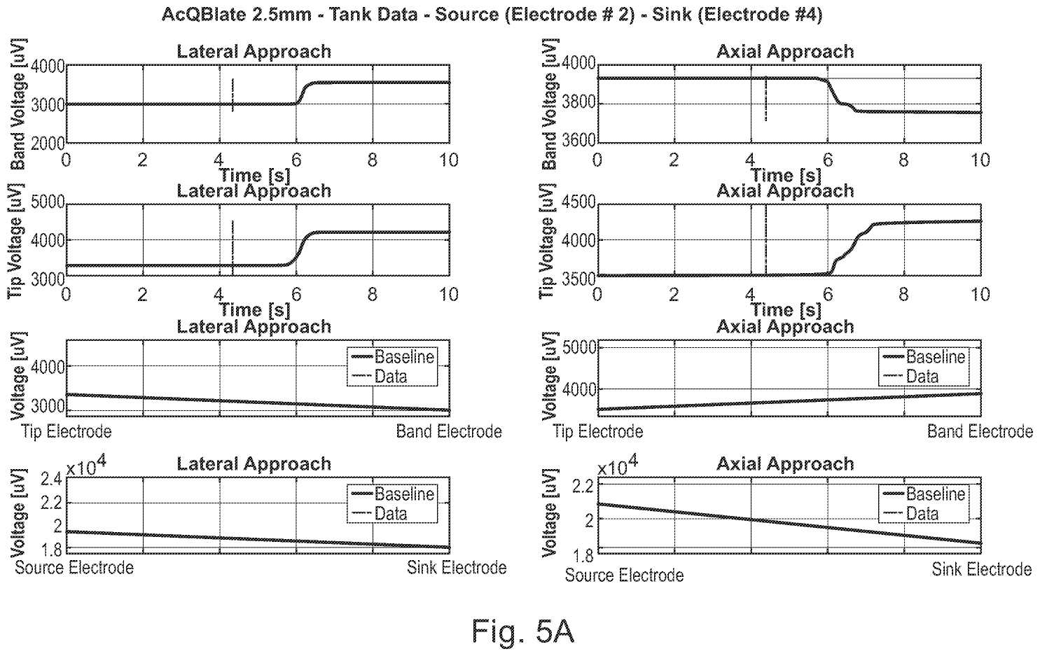

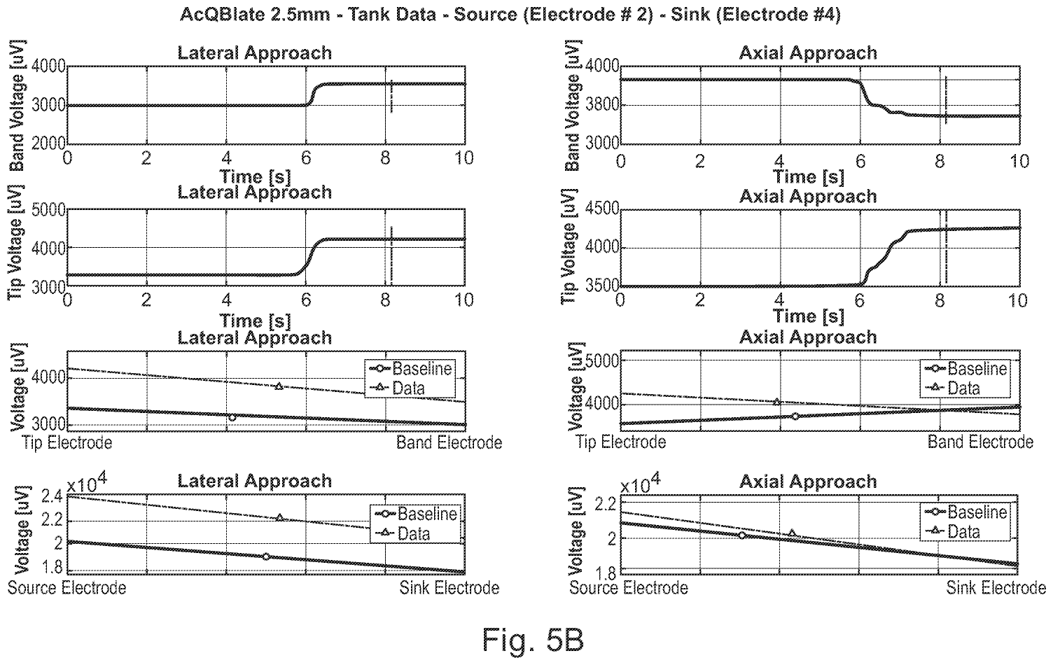

Referring now to FIG. 5, a graph of voltages measured from electrodes of an ablation catheter is illustrated, consistent with the present inventive concepts. In some embodiments, console 100 is configured to estimate the angle between a portion of ablation catheter 200 and the cardiac wall. The electric field created by source and sink electrodes (e.g., electrodes 232b and 232c) of ablation catheter 200 is dependent on the location and orientation of catheter 200 with respect to the cardiac wall. This electric field distorts as catheter 200 approaches the wall. This field distortion that results when the catheter approaches axially, laterally, or obliquely (e.g., approximately 45°) differs in the different approaches, and system 10 can identify the particular approach orientation based on an analysis of this field distortion. FIG. 5 illustrates a baseline (dashed) representing data recorded when catheter 200 is not in contact with the cardiac wall, as well as a “Tip-band voltage line” (solid line) representing real time data between voltage measured by tip electrode 231 and a first ring electrode 232a of ablation catheter 200. In some embodiments, the relative orientation between these two lines can estimate an angle θ, where angle θ is the angle between the axis of ablation catheter 200 and the tangent vector of the surface of the cardiac wall. Plotting the measured voltage along the axis of catheter 200 can indicate the relative change of the tip-band voltage line with respect to the baseline. In some embodiments, this plot can be used (e.g., by system 10 and/or an operator of system 10) to determine the orientation of catheter 200 relative to the cardiac wall. For example, the vertical intercept of the tip-band voltage line, as well as the slope of the tip-band voltage line, changes as catheter 200 approaches the tissue, and this change can be used by system 10 to determine how catheter 200 is oriented with respect to the cardiac surface. In some embodiments, angle θ can be defined by the following function:

θ=ƒ(V_{intercept},m)

In some embodiments, function ƒ of the above equation can be found (e.g., found via testing, such as in a calibration or manufacturing process) by placing catheter 200 in known orientations relative to a model of tissue, for example, at angles of 0° (lateral), 22°, 45°, 67°, and 90° (axial) and by measuring the intercept and the slopes as shown in FIG. 5, and then by fitting the best function to this data to determine the relationship between V_{intercept}, m, and θ.

Referring additionally to FIGS. 5A and 5B, various graphs of test data collected by the applicant are illustrated, consistent with the present inventive concepts. The data illustrates how the lateral, θ=0°, and axial, θ=90°, approaches have distinct features in terms of intercept and slope changing with respect to the baseline (non-contact state). The data was gathered as a test catheter approached the test medium (e.g., dead beef tissue) laterally and axially until contact was made without compressing the piston. The lateral approach showed the tip-band voltage line offsets from the baseline in a parallel fashion whereas that of the axial approach tilts and intersects the baseline.

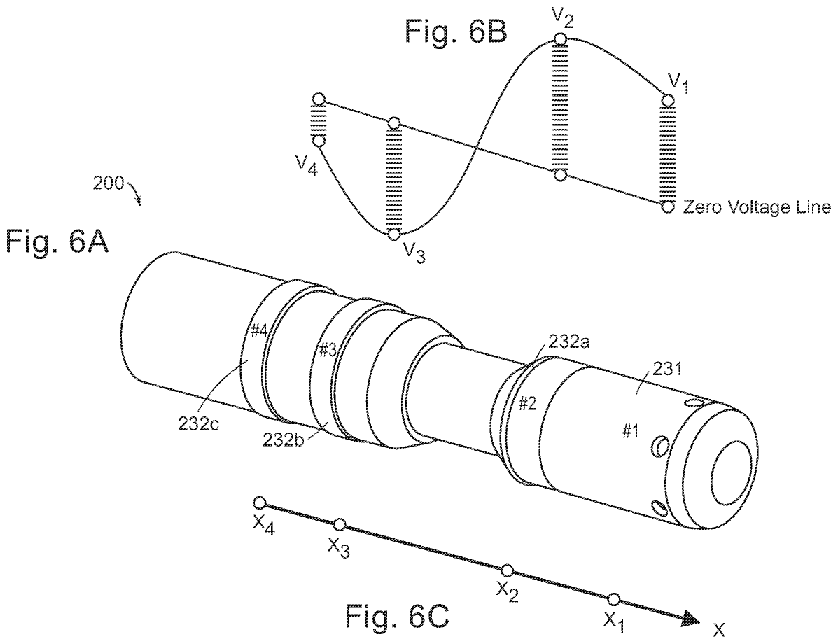

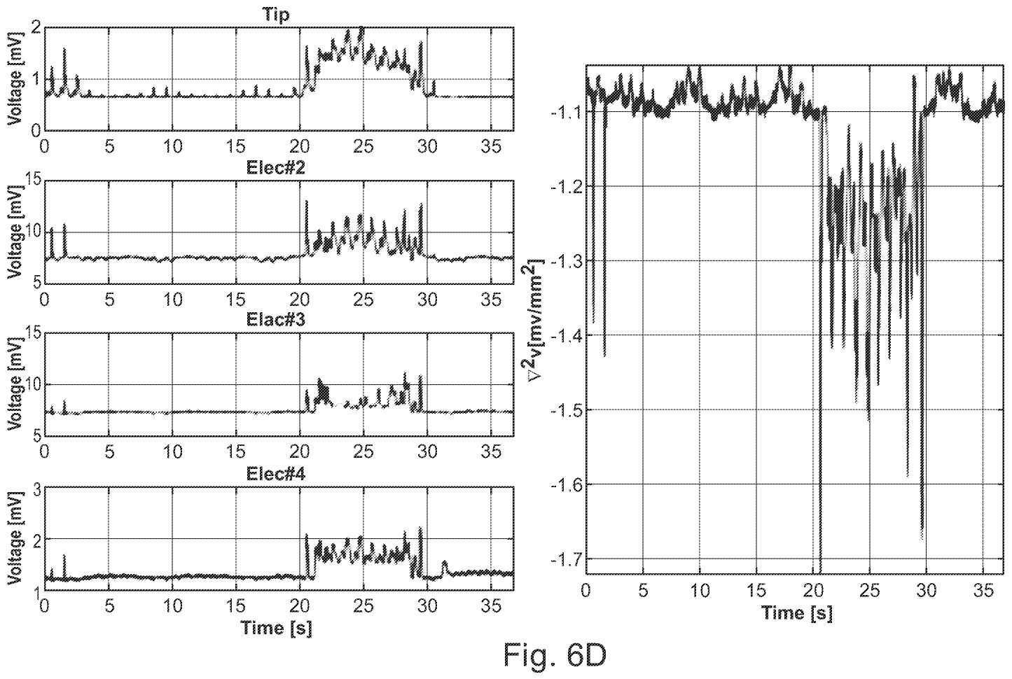

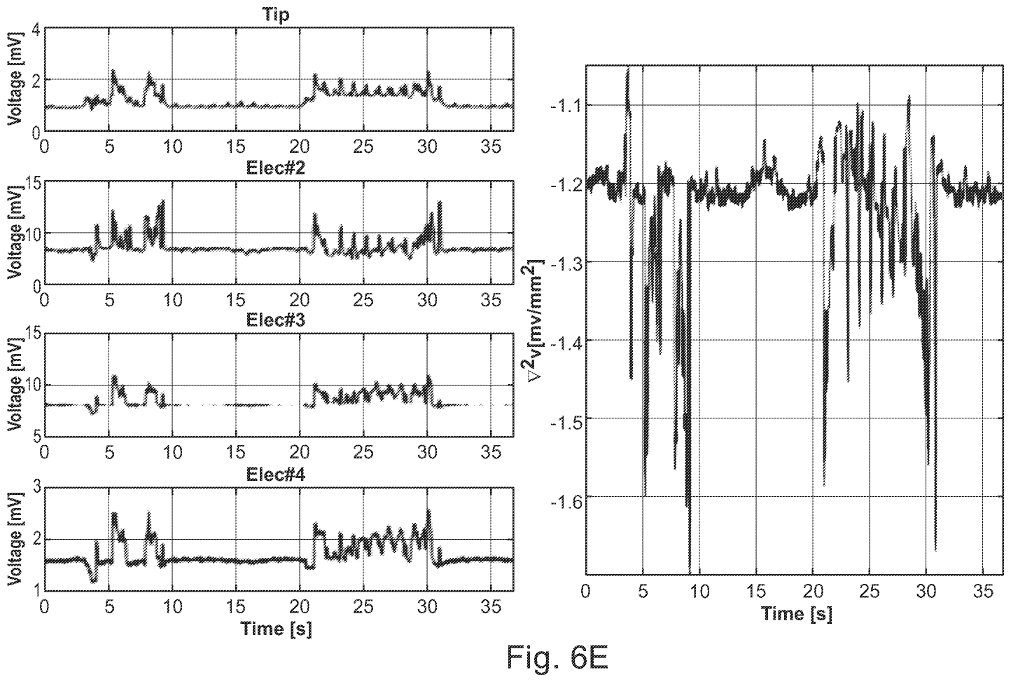

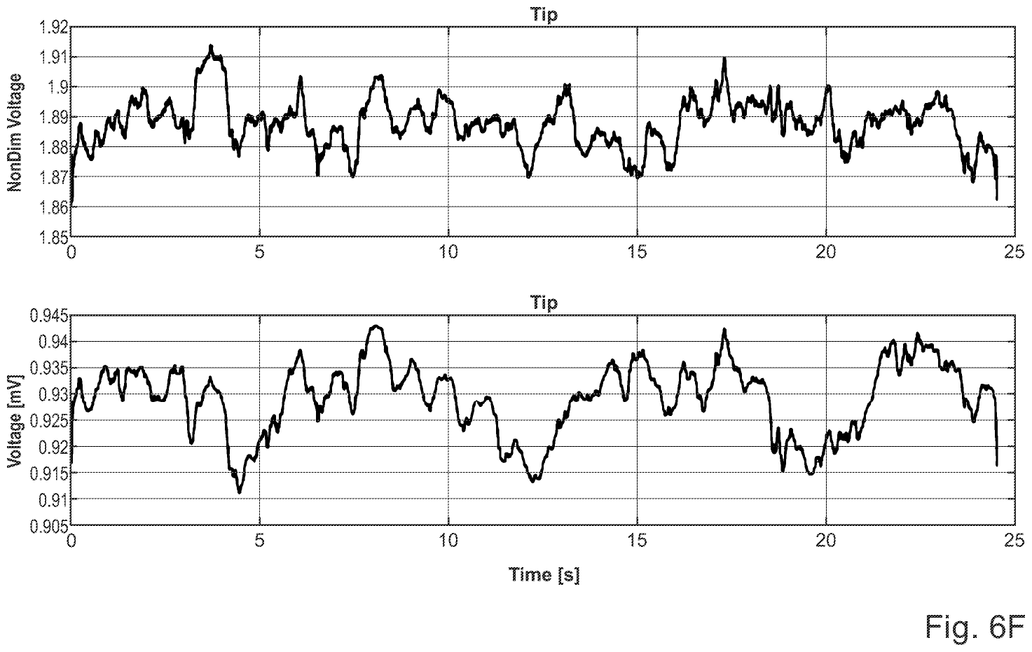

Referring now to FIGS. 6A-C, a distal portion of an ablation catheter, a position indicating line, and a graph of voltage are illustrated, respectively, consistent with the present inventive concepts. FIGS. 6D-F illustrate various graphs of recorded electrode voltages and calculated data, consistent with the present inventive concepts. In some embodiments, console 100 is configured to determine if a portion of ablation catheter 200 is in contact with the cardiac wall, such as is described herein. This contact determination can be based on one or more measurements that are dependent on the conductivity of the blood of the patient. In some embodiments, console 100 is configured to provide a signal to one or more electrodes of ablation catheter 200 (e.g., a signal between electrodes 232a and 232b, and or between an electrode 232 and one or more body surface electrodes such as patch 60), to measure the voltages that result from that signal, and to determine one or more characteristics based on the voltage measurements. The one or more characteristics can include a mathematically determined quantity, κ, described herebelow. FIG. 6A illustrates the distal portion of ablation catheter 200, and FIG. 6B illustrates a representation of a signal which is sourced and sinked between electrodes 232a and 232b. Quantity κ can be defined as follows:

$$κ = \frac{{\partial }^{2} V}{\partial {x}^{2}} {\vert}_{Elec \#2} = 2 [ \frac{{V}_{3} - {V}_{2} ( 1 + α ) + {αV}_{1}}{α ( 1 + α ) {h}^{2}} ] \qquad ( B1 )$$

Using the position indicators of FIG. 6C, in equation B1, x is the coordinate along the axis of the catheter 200; V₁, V₂, V₃ are the voltage values at the tip, source, and sink electrodes in a 2-3 configuration (e.g., a configuration where the signal is sourced from electrode 232a and sinked to electrode 232b); h=x₂−x₁ is the distance between the tip electrode to the second electrode (center-to-center); and α=(x₃−x₂)/(x₂−x₁) is the ratio of the distances among electrodes 1, 2, and 3. In the 2-3 configuration shown, electrode number 3 is the sink, and as such V₃ must be a negative number in equation B1. Quantity κ is representative of the voltage profile shape along catheter 200 (voltage profile curvature), and can be used to: estimate the electrical conductivity of blood; scale V₁, V₂, V₃, V₄ as if they are measured in a reference conductivity (e.g., 7.0 mS/cm) detect contact state of one or more electrodes of catheter 200; filter (e.g., reduce) respiration effects (e.g., when κ is implemented in a filter); and combinations of these.

For example, κ can be used in estimating the electrical conductivity of blood using the following equations. The governing equation for conservation of current is the following Poisson's equation:

σ∇²V=−Q− [symbol] · [symbol] (B2)

where V is voltage potential, σ is the electrical conductivity, Q is the current density. When ablation catheter 200 is far from a surface boundary (e.g., in a baseline condition, with no-contact, and ablation catheter 200 is placed at the center of atrium), equation B3 results as follows:

[symbol]

=0 (B3)

and therefore,

σ∇²V=−Q (B4)

and solving for conductivity yields:

$$σ = \frac{- Q}{{∇}^{2} V} \qquad ( B5 )$$

Given that the amount of current going to ablation catheter 200 should remain constant, Q=const., electrical conductivity becomes only a function of Laplacian of voltage across ablation catheter 200, which is patient-specific. In a cylindrical coordinate system, equation B6 is as follows:

$${∇}^{2} V = \frac{1}{r} \frac{\partial }{\partial r} ( r \frac{\partial V}{\partial r} ) + \frac{1}{{r}^{2}} \frac{{\partial }^{2} V}{\partial {θ}^{2}} + \frac{{\partial }^{2} V}{\partial {x}^{2}} \qquad ( B6 )$$

Due to angular symmetry, equation B7 is as follows:

$$\frac{{\partial }^{2} V}{\partial {θ}^{2}} = 0 \qquad ( B7 )$$

At radial distances very close to the catheter, the Laplacian can be approximated by:

$${∇}^{2} V \sim \frac{{\partial }^{2} V}{\partial {x}^{2}} \qquad ( B8 )$$

∂²V/∂x² in a non-uniform spacing among the first three electrodes which can be found by:

$$\frac{{\partial }^{2} V}{\partial {x}^{2}} {\vert}_{Elec \#2} = 2 [ \frac{{V}_{3} - {V}_{2} ( 1 + α ) + {αV}_{1}}{α ( 1 + α ) {h}^{2}} ] \qquad ( B9 )$$

where h=x₂−x₁, α=(x₃−x₂)/(x₂−x₁).

As another example of the uses of κ, a scaling factor to scale V₁, V₂, V₃, V₄ can be determined as if they are measured in a reference conductivity. In two different electrical conductivities, the voltages measured by 4 electrodes are different. For example, ablation catheter 200 can be assumed in two conductivities σ≠σ_{ref}. The following scale factor can be used to scale the measurement to the reference conductivity.

$$s = \frac{σ}{{σ}_{ref}} = \frac{{( {∇}^{2} V )}_{ref}}{{∇}^{2} V} \qquad ( B10 )$$

In some embodiments, κ can be used to detect a contact state of ablation catheter 200. ∇²V can also be used for contact state detection due to its different behavior under different contact states, as shown in FIGS. 6D and 6E. FIG. 6D illustrates data gathered by the applicant related to lateral contact between an ablation catheter 200 and in-vivo swine tissue. In the graphs of FIG. 6D, electrode voltages are shown in the left column, and the Laplacian of voltage is shown in the right column. FIG. 6E illustrates data gathered by the applicant for axial contact between an ablation catheter 200 and in-vivo swine tissue. In the graphs of FIG. 6E, electrode voltages are shown in the left column, and the Laplacian of voltage is shown in the right column.

FIG. 6F illustrates an example of respiration removal effect of κ. The illustrated data was gathered by applicant. The two graphs illustrate tip electrode 231 voltage, recorded when catheter 200 was in no-contact state (otherwise referred to as baseline) condition. In the bottom graph, the original tip electrode voltage has been smoothed by a median filter for illustrative purposes. In the top graph, non-dimensional tip electrode voltage V*_{Tip}=V_{Tip}/κ is illustrated.

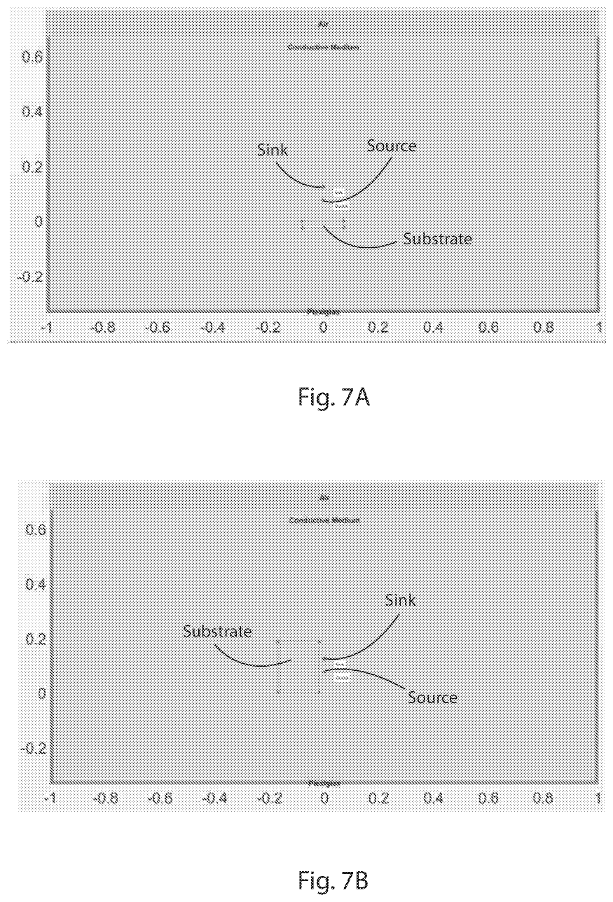

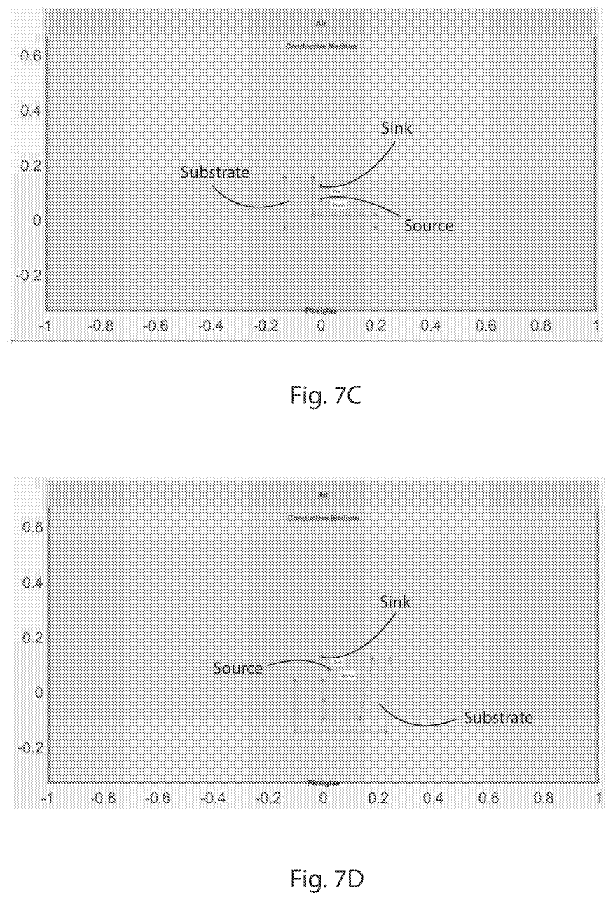

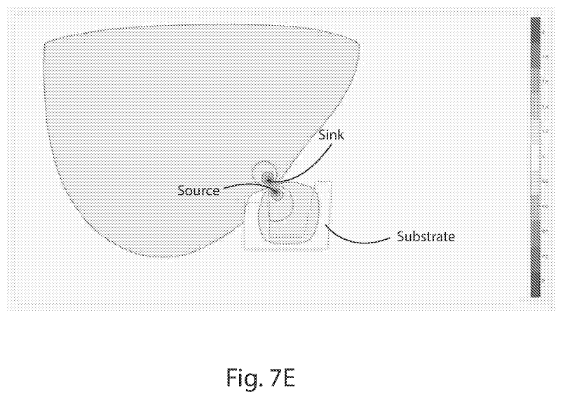

Referring now to FIGS. 7A-E, various simulation schematics and results are illustrated, consistent with the present inventive concepts. Applicant has performed various simulations modeling various electric fields surrounding an ablation catheter 200 as it comes into contact with tissue in different orientations. By simulating the potential field surrounding an ablation catheter 200, the applicant gained insight into design, optimization, and understanding of the catheter 200 behavior when implementing contact sensing technology, such as is described herein. The electric field-based contact sensing technology of system 10 as described herein can be heavily dependent on the voltage change to differentiate contact versus non-contact states. Since the potential field is affected by the orientation of the source and sink electrodes of catheter 200, as well as the proximity with respect to the cardiac wall, it was important to study the field change in different arrangements to learn about the performance of the ablation catheter 200, which informed algorithm development (e.g., the development of algorithm 115).

Applicant has developed an interactive MATLAB interface for 2D modeling of the potential field of the catheter inside of a test tank (e.g., a tank filled with a conductive fluid configure to simulate the conductivity of blood). The interface created a computational domain that represented a tank made of acrylic, filled with a saline solution, and comprising substrates of various shapes, such that the applicant could model a catheter being exposed to different boundary conditions (e.g., different contact force and angle of contact conditions). The various shapes of the substrates allowed the applicant to simulate numerous edge conditions that the catheter 200 can experience. The substrate was able to be interactively drawn to any shape. The interface was configured to discretize and pixelate the domain into small areas and assigns the proper permittivity to different mediums. It then used a Poisson solver to obtain the potential field around the source and sink electrodes of the simulated catheter. This tool was used to perform a quasi-static simulation of a catheter in force maintenance condition.

In FIG. 7A, the simulated substrate is illustrated, and the source and sink electrodes are positioned in an axial orientation with respect to the substrate.

In FIG. 7B, the simulated substrate is illustrated, and the source and sink electrodes are positioned in a lateral orientation with respect to the substrate.

In FIG. 7C, the simulated substrate is illustrated, and the source and sink electrodes are positioned in an axial orientation with respect to one surface of the substrate and in a lateral orientation with another surface of the substrate.

In FIG. 7D, the simulated substrate is illustrated, and the source and sink electrodes are in a complex orientation with respect to the substrate.

In FIG. 7E, the result of the simulated orientation illustrated in FIG. 7D is illustrated.

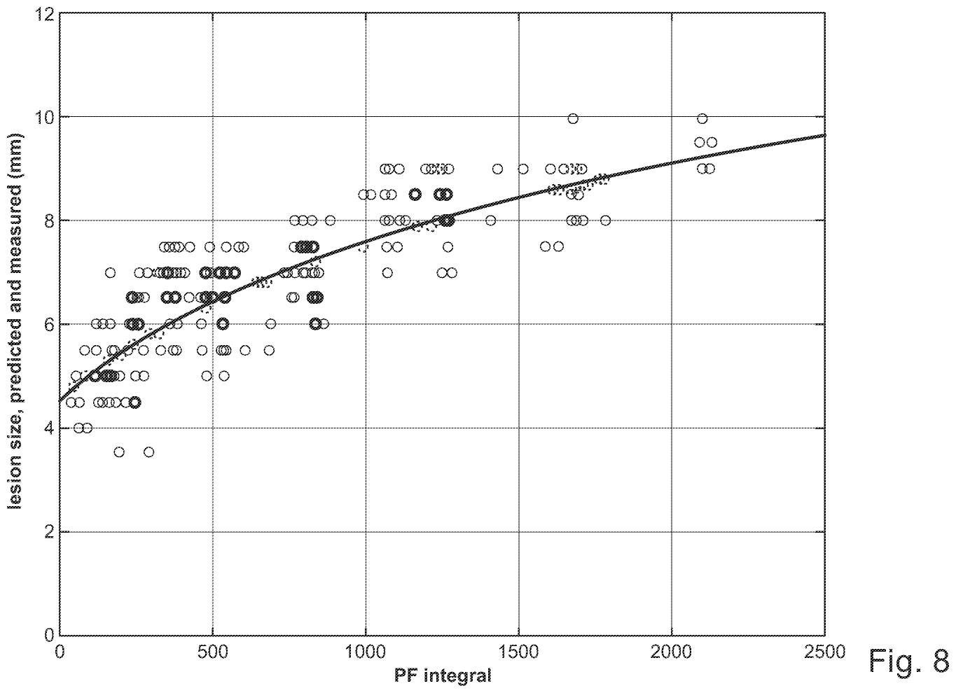

Referring now to FIG. 8, a graph of in-vitro study data is illustrated, consistent with the present inventive concepts. In some embodiments, algorithm 115 includes a predictive model for lesion size based on power, contact force, and time. The predictive model can be described as follows:

$${W}_{p} = α ln ( β \hat{P} + 1 ) + γ \\ \hat{P} = \int P \hat{F} dt \\ \hat{F} = \{ \begin{matrix}\frac{F}{10} : & F \leq 10 \\ 1 : & F > 10\end{matrix}$$

where F is in gm, and power, time, and lesion size are in units consistent with input data. The model includes three empirical constants that are determined by a non-linear recursive least squares method, α, β, and γ. The in-vitro data illustrated in FIG. 8 was experimentally obtained by the applicant using bovine ventricle tissue, with power measured in Watts, force measured in grams (or gram force (gmf)), time measured in seconds, and the average lesion width measured in millimeters. In the graph of FIG. 8, the blue points illustrate the experimental data, the line is a LS fitted function, and the green points represent a prediction for in-vivo data. Multiple values of the three constants described hereabove may be applicable depending on the nature and quality of the input data.

The above-described embodiments should be understood to serve only as illustrative examples; further embodiments are envisaged. Any feature described herein in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the inventive concepts, which is defined in the accompanying claims.

1. A system for performing a medical procedure on a patient, comprising:
an ablation catheter comprising:
a shaft;
at least one therapeutic element positioned on the shaft; and
one or more tissue-contact sensing components configured to provide a contact signal related to the level of contact between the at least one therapeutic element and tissue;
an energy delivery unit configured to deliver ablative energy to the at least one therapeutic element to create a lesion in target tissue, and to provide an energy delivery data related to one or more parameters of the delivered energy;
a force module configured to receive the contact signal and provide contact data related to the level of contact between the at least one therapeutic element and tissue; and
a processing unit configured to receive the energy delivery data and the contact data and to determine an ablation index based on both the energy delivery data and the contact data,
wherein the ablation index represents the status of the lesion being created, and
wherein the ablation index is based on the following calculations:
W(t,\hat{P},\hat{F})=κ+W_{∞} ln(αt+1)ln(β\hat{P}+1)(1−exp(−γ\hat{F})),
with
$$\hat{P} = {\int }_{{t}_{0}}^{T} P ( t ) dt , and \\ \hat{F} = \frac{1}{( T - {t}_{0} )} {\int }_{{t}_{0}}^{T} F ( t ) dt or \hat{F} = {\int }_{{t}_{0}}^{T} F ( t ) dt ,$$
where t₀ is time at the start of ablation.

2-36. (canceled)
